SEQUENCE LISTING (11 sequences)

SEQ ID NO: 1 - DNA, 15 bp, Artificial Sequence
  Polynucleotide (peptide nucleic acid)
gtcccguccccgcca

SEQ ID NO: 2 - protein, 8 aa, Artificial Sequence
  Synthetic Peptide
LPXTAAAA

SEQ ID NO: 3 - DNA, 28 bp, Artificial Sequence
  Polynucleotide
tcaagtagcttcagaatcttttcccccc

SEQ ID NO: 4 - DNA, 15 bp, Artificial Sequence
  Polynucleotide
ggctagctacaacga

SEQ ID NO: 5 - RNA, 17 nt, Artificial Sequence
  Antisense
gucccugucgggcgcca

SEQ ID NO: 6 - RNA, 18 nt, Artificial Sequence
  Antisense
cagggacaagcccgcggu

SEQ ID NO: 7 - protein, 5 aa, Artificial Sequence
  Synthetic Peptide
LPXTG

SEQ ID NO: 8 - DNA, 669 bp, Artificial Sequence
  (I-A) HS-LPGTG  Variable portion of the heavy chain of the anti-HEL (Hen Egg Lysozyme) antibody
caggtgcagctgaaagaatctggtccgggtctggtggcgccgtctcagtctctgtctattacctgcaccgtgtctggtttttctctgaccggttatggtgtgaactgggtgcgtcagccgccgggtaaaggtctggaatggctgggtatgatttggggtgatggtaacaccgattataactctgcgctgaaatctcgtctgtctatttctaaagataactctaaatctcaggtgtttctgaaaatgaactctctgcataccgatgataccgcgcgttattattgcgcgcgtgaacgtgattatcgtctggattattggggtcagggtaccaccctgaccgtgtcttctgcgtctaccaccccgccgtctgtgtttccgctggcgccgggttctgcggcgcagaccaactctatggtgaccctgggttgcctggtgaaaggttattttccggaaccggtgaccgtgacctggaactctggttctctgtcttctggtgtgcatacctttccggcggtgctgcagtctgatctgtataccctgtcttcttctgtgaccgtgccgtcttctccgcgtccgtctgaaaccgtgacctgcaacgtggcgcatccggcgtcttctaccaaagtggataaaaaaattgtgccgcgtgattgcctgccgggtaccggt

SEQ ID NO: 9 - DNA, 657 bp, Artificial Sequence
  (I-B) LS-LPGTG  Variable portion of the light chain of the anti-HEL (Hen Egg Lysozyme) antibody
gatattcagatgacccagtctccggcgtctctgtctgcgtctgtgggtgaaaccgtgaccattacctgccgtgcgtctggtaacattcataactatctggcgtggtatcagcagaaacagggtaaatctccgcagctgctggtgtattataccaccaccctggcggatggtgtgccgtctcgtttttctggttctggttctggtacccagtattctctgaaaattaactctctgcagccggaagattttggttcttattattgccagcatttttggtctaccccgcgtacctttggtggtggtaccaaactggaaattaaacgtgcggatgcggcgccgaccgtgtctatttttccgccgtcttctgaacagctgacctctggtggtgcgtctgtggtgtgctttctgaacaacttttatccgaaagatattaacgtgaaatggaaaattgatggttctgaacgtcagaacggtgtgctgaactcttggaccgatcaggattctaaagattctacctattctatgtcttctaccctgaccctgaccaaagatgaatatgaacgtcataactcttatacctgcgaagcgacccataaaacctctacctctccgattgtgaaatcttttaaccgtaacgaatgcctgccgggtaccggt

SEQ ID NO: 10 - RNA, 18 nt, Artificial Sequence
  “Primer Binding Sequence” RNA (or PBS) from HIV-1
uggcgcccgaacagggac

SEQ ID NO: 11 - DNA, 63 bp, Artificial Sequence
  CCR5-WT (775-837)
agctctcattttccatacagtcagtatcaattctggaagaatttccagacattaaagatagtc

(19) United States
(12) Patent Application Publication
(10) Pub. No.: US 2013/0101665 A1
(43) Pub. Date: Apr. 25, 2013

(54) SELF-ASSEMBLING HALF-ANTIBODIES

(71) Applicants: Nicolas Ugolin, Paris (FR); Caroline Falck, Clamart (FR); Emilie Lefevre, Fontenay Aux Roses (FR); Sylvie Chevillard, Paris (FR); Carine Tisne-Vicrobeck, Levallois Perret (FR)

(72) Inventors: Nicolas Ugolin, Paris (FR); Caroline Falck, Clamart (FR); Emilie Lefevre, Fontenay Aux Roses (FR); Sylvie Chevillard, Paris (FR); Carine Tisne-Vicrobeck, Levallois Perret (FR)

(73) Assignee: CENTRE NATIONAL DE LA RECHERCHE SCIENTIFIQUE, Paris (FR)

(21) Appl. No.: 13/640,202

(22) PCT Filed: Apr. 8, 2011
(86) PCT No.: PCT/FR2011/050812

§ 371 Date: Dec. 20, 2012

(30) Foreign Application Priority Data
Apr. 9, 2010 (FR) 10/01516

(51) Int. Cl. C07K 16/00

(52) U.S. Cl. CPC C07K 16/00
USPC 424/450; 530/387.3; 424/133.1; 435/6.12; 506/18; 506/9; 506/16; 435/199

(57) ABSTRACT

Novel chimeric molecules, termed “half-antibodies”, which are capable of self-assembling to form an epitope recognition site. Using these half-antibodies or a vesicle, a viral particle, a composition or a kit thereof, for therapeutic applications, such as the prevention or treatment of cancers, genetic diseases, infectious diseases, and for in vitro diagnostic applications and detecting biological molecules. The half-antibodies include at least two chimeric molecules A and B, each has a polypeptide domain characteristic of a variable domain of a heavy chain or of a light chain of an antibody, and a nucleotide domain, the nucleotide domain of A and that of B being capable of pairing into a double stranded structure. Biologically active nucleic sequences can be grafted onto these chimeric molecules to prevent the expression of target genes in the interior of a human or non-human mammalian cell.

The invention relates to novel chimeric molecules, termed “half-antibodies”, which are capable of self-assembling to form an epitope recognition site. A half-antibody is constituted by at least two chimeric molecules A and B, each comprising a characteristic polypeptide domain of a variable domain (or VD) of a heavy chain or of a light chain of an antibody, and a nucleotide domain, the nucleotide domain of A and that of B being capable of pairing into a double stranded structure, for example in a hydric medium. Biologically active nucleic sequences can be grafted onto such chimeric molecules in order, for example, to prevent the expression of target genes inside a human or non-human mammalian cell.

The invention also relates to various applications of these half-antibodies, in particular the use of these half-antibodies for therapeutic treatment or for in vitro diagnostics, and more generally to the detection of biological molecules.

Said half-antibodies are of great potential on the one hand for the treatment of genetic diseases or cancers and on the other hand for the treatment of infectious diseases. In the context of certain genetic diseases, such as myopathy, using such a chimeric molecule can prevent the expression of mutated genes at the translation stage, in order to reduce the presence of defective proteins that are at the origin of the disease. In the field of the fight against cancer, this chimeric molecule can be used to inactivate intra-cellular proteins. For infectious diseases (in particular viral diseases and bacterial infections), this chimeric molecule can also be used to block the expression of genes of the pathogenic agent that specifically condition either the primary activities of the pathogenic agent (for example, retro-transcription in a retrovirus) or resistances to existing treatments (for example the resistance of bacteria to antibiotics).

Nucleic acids or nucleic acid analogs, which may be antisense or have a ribo- or deoxyribonuclease activity, are used to block the expression of genes either at the replication, transcription or translation stage. Because they can act directly on a target sequence, those sequences have great potential in the treatment of genetic diseases, infectious diseases, and in the fight against resistance mechanisms (for example the resistance of bacteria to antibiotics).

However, the use of nucleotide sequences or analogs as a drug has been observed to be considerably limited, on the one hand because of the necessity of specifically addressing these sequences to the target cell and on the other hand by the fact that such sequences do not pass efficiently through the cellular membrane.

In order to improve their efficiency of passage through the cellular membrane, such nucleotide sequences or analogs can be modified by adding, at the 5′ or 3′ end, lipophilic chemical groups or functions (Manoharan et al., 1995; Pfeiffer and Höök, 2004) or short peptides, generally constituted by 1 to 5 beta-amino acid residues (i.e. amino acids in which the amine group is in the beta (β) position with respect to the carboxylate group). That modification can facilitate the passage of nucleic acids through the plasma membrane, as has been demonstrated on cell cultures (Tripathi et al., 2005).

Although passage through the plasma membrane is facilitated, simply adding a hydrophobic group at the end of the nucleic sequences, however, does not allow them to be used in the context of therapeutic treatments because of the non-specific nature of hydrophobic groups. In fact, nucleic acid sequences modified in that manner are mostly to be found absorbed in an aspecific manner by various cells before they reach the target, thus reducing the effect of the treatment.

Thus, specific addressing of such molecules to the target is necessary.

In order to overcome those addressing problems irrespective of the medium conditions, the inventors have developed chimeric molecules comprising a nucleic domain, for example DNA, and a polypeptide domain characteristic of an antibody fragment comprising an epitope recognition side.

These nucleic acid/antibody chimeric molecules (for example DNA/antibody or DNA/AB) may be used to tag and amplify protein markers, for example on the surface of a cell, or to functionalize a surface with antibodies in the context of producing biochips.

The prior art discloses antibody/nucleic acid chimeric molecules (FR286006; FR2882563; FR2882564), but they suffer from disadvantages:

a) the mechanism for penetration into a cell is based on cellular pinocytosis or endocytosis, which involves a non-covalent interaction between the nucleic acid and the antibody, such that the change in pH after fusion of endocytosis vesicles with basic vesicles or intracellular acids ruptures the non-covalent interactions and separates the nucleic acids from the antibodies. This mode of addressing and absorption involves cellular activity and thus is not independent of the state of the cell. That embodiment will not be effective, or only be slightly effective on cellular models carrying out little or no endocytosis;

b) the nucleic acid sequences that are already used in the antibody/nucleic acid molecules are generally antisense in type and possibly micro RNA (miRNA) destined for proteosome systems; they do not have enzymatic activity per se (Baulcombe, 2002);

c) in addition, grafting nucleotide sequences onto antibodies that have already been constituted is limited on the one hand by the poor reproducibility of the graft site on the antibody (Fan et al., 2008) and on the other hand by maintaining the antibody recognition site; there is in fact a substantial probability that functionalization by the nucleic acid will occur in the active site of the antibody, thereby having a negative influence on its activity;

d) producing antibody from cloned sequences is still very difficult. An antibody is a complex assembly of polypeptide chains bonded via disulfide bridges. It is also a relatively heavy protein (on average 150 kDa) and in vitro production using cloning and recombinant expression methods remains difficult. In fact, once the chains have been produced, the step for re-constitution by pairing between those light chains and heavy chains and establishing the correct disulfide bridges is still a limiting step. A wide variety of incorrect pairings are formed between the chains; of the combinations of heavy/light chains obtained, a large proportion is not functional because of improper pairing of these chains.

In order to overcome these various disadvantages, the inventors have developed particular chimeric molecules, termed “half-antibodies” (or “HAB”), which comprise characteristic polypeptides of variable portions of the antibody, and nucleic acids (or analogs) that are capable of self-assembling to re-form a recognition site of an antibody that is functional in an aqueous medium, including in a reducing medium.

The delivery of nucleic sequences of interest (in particular biologically active nucleic sequences) into target cells via the chimeric molecules developed by the inventors is partly independent of the endocytosis and/or pinocytosis mechanism and thus can be used to target any cell independently of its metabolism.

Thus, the invention provides a chimeric molecule, termed a “half-antibody”, characterized in that it comprises or consists of two chimeric molecules A and B, each comprising or consisting of:

(i) a characteristic polypeptide domain of a variable domain (or VD) of a heavy chain or of a light chain of an antibody, this polypeptide domain being positioned at one end in the chimeric molecules A and B, the polypeptide domain (i) of one of the two chimeric molecules, A or B, being characteristic of a VD of a light chain of an antibody and the polypeptide domain (i) of the other chimeric molecule, respectively B or A, being characteristic of a VD of a heavy chain of an antibody (the same antibody or a distinct antibody); and

(ii) a nucleotide domain (or structural domain) comprising or consisting of a polynucleotide, in particular a DNA or a RNA, or an analog of a polynucleotide, in particular a peptide nucleic acid (PNA), a locked nucleic acid (or LNA), a methylphosphonate nucleic acid, or a thioate nucleic acid (in particular a phosphorothioate or phosphorodithioate nucleic acid), the nucleotide domain of A and that of B being capable of pairing into a double stranded structure, for example in a hydric medium, in particular in a reducing medium; and

(iii) if appropriate, one or more linkers, in particular a linker that binds domains (i) and (ii) of a chimeric molecule A or B, said linker(s) comprising or consisting of a hydrocarbon chain (or backbone) preferably comprising 1 to 20 carbon atoms, the domains (i) and (ii) of each of the chimeric molecules A and B being bonded via a covalent bond, for example via a NH₂ group, in particular a NH₂ group of a linker.

Unless indicated otherwise, each particular embodiment presented in the present application may be independently combined with one or more other particular embodiments presented in the application.

The half-antibody of the invention is capable of self-assembling, in particular in a hydric or aqueous medium, including a reducing medium, via the domain (ii) of the chimeric molecule A and that of the chimeric molecule B, which pair together into a double stranded structure.

The term “chimeric” as used here denotes a molecule that associates a plurality of domains of at least two different types in their function and/or their nature, at least two of said domains being of a distinct nature, one being of a polypeptide nature and the other being of a nucleotide nature.

The term “more” as used in the present application means two or more than two (for example three, four, five, six or seven).

The term “half-antibody” as used here means a particular chimeric molecule comprising or consisting of:
at least one peptide component characteristic of an antibody, which comprises or consists of an epitope recognition site (or paratope), i.e. capable of binding to an epitope; and
a nucleotide component (for example a double stranded nucleotide structure), which stabilizes the chimeric molecule.

The term “variable domain” (or “VD”) as used in the present application means a polypeptide that constitutes a portion of a recognition site of an epitope, i.e. a polypeptide involved in recognition of an epitope and in the interaction with said epitope. In a particular embodiment of the invention, said VD comprises or consists of:
the variable domain V_{H} carried by the heavy chain of an antibody (i.e. a variable domain coded by a gene comprising the segments V (variable), J (junction) and D (diversity)); or
the variable domain V_{L} carried by the light chain of an antibody (i.e. a variable domain coded by a gene comprising segments V and J but no segment D); or
one or more portion(s) of V_{H} or V_{L}, in particular one or more portion(s) of V_{H} or V_{L} that is (are) capable of binding to said epitope, said VD conserving the capacity of V_{H} or V_{L} to interact with said epitope.

By way of example, in a particular embodiment of the invention, the expression “portion(s)” of V_{H} or V_{L} means one or more (in particular two, three or even four) hypervariable regions, also termed complementary determining regions (CDRs), and in particular all CDRs of a V_{H} or V_{L} domain. Said CDR regions are, if appropriate, used in combination with non-variable fragments (frameworks 1, 2 etc.) present in the native variable domains of the antibodies.

The expression “characteristic polypeptide domain of a VD” as used here means a polypeptide domain having the properties of a VD domain, i.e. a polypeptide domain involved in recognition of an epitope and in binding to said epitope. Said polypeptide domain may be of synthetic origin or be obtained from or derived from a VD of a given antibody (for example a human or humanized antibody), in particular, said polypeptide domain may comprise or consist of a VD of a given antibody (for example a human or humanized antibody), or consist of a sequence obtained by insertion, deletion or substitution of amino acid residues, for example one, two, three, four or five residues, in the sequence of a VD of a given antibody.

In accordance with a particular embodiment, said polypeptide domain derives from a VD obtained by substitution of amino acid residues, for example one, two, three, four or five residues, by a monomer residue of a PNA or a beta-amino acid (for example a beta-alanine). This in particular means that the possibilities of recognition of said VD can be increased. When the chimeric molecules A and B of a half-antibody are assembled (via their respective nucleotide domain (ii)), the polypeptide domain (i) of A and that of B constitute an epitope recognition site that is capable of interacting (preferably specifically) with an epitope.

A half-antibody of the invention may be obtained by recombining fragments of different antibodies and in particular VD domains, or their portions that are active in recognition of an epitope, obtained from or derived from different antibodies, said different antibodies possibly being obtained from the same human or non-human mammalian species. In a particular embodiment of the invention, a half-antibody comprises at least two VDs obtained from or derived from given antibodies (from one and the same antibody or from distinct antibodies), in particular a VD positioned at the N-terminal end of the chimeric molecule A and a VD positioned at the N-terminal end of the chimeric molecule B, which on assembling and becoming structured together form an epitope recognition site.

In a particular embodiment of the invention, the domain (i) of the chimeric molecule A or B of a half-antibody of the invention comprises or consists of the variable V_{H} domain of an antibody or one or more portion(s) of the V_{H} domain of an antibody and the domain (i) of the other chimeric molecule of said half-antibody (respectively B or A) comprises or consists of the variable domain V_{L} of the same antibody or of a distinct antibody, or one or more portion(s) of this domain V_{L} (said portions of V_{H} or V_{L} being as defined in the application).

In a particular embodiment of the invention, the domains (i) of molecules A and B of a half-antibody are humanized.

The term “at least x” element(s) as used in the present application means x element(s) or more than x element(s), for example two, three or four elements.

The expression “phosphonate nucleic acid” or “thioate nucleic acid” as used here means modified nucleic acids in which the phosphorus atoms are blocked (i.e. esterified with methanol) or substituted with sulfur.

A polypeptide domain (i) may be characteristic of a VD of a heavy chain or of a light chain of any antibody.

In a particular embodiment of the invention, the polypeptide domain (i) of the chimeric molecule A and that of B are characteristic of a VD of the same antibody or, in contrast, of distinct antibodies.

In a particular embodiment of the invention, the polypeptide domains (i) of A and of B comprise at least one cysteine that can be used to establish a disulfide bridge between the polypeptide domain (i) of A and that of B, and in particular at least one cysteine that can be used to establish a disulfide bridge between the VD of A and that of B.

In a particular embodiment of the invention, the polypeptide domains (i) of A and of B are characteristic of a VD of an antibody recognizing (preferably specifically) an epitope of an intra- and/or extra-cellular antigen, more particularly of a surface antigen, for example an antigen expressed at the surface of organelles or a cellular or intra-cellular surface antigen. These antigens may in particular be of cellular, viral, bacterial or tumoral origin.

In a particular embodiment of the invention, the polypeptide domains (i) of A and of B, in particular their N-terminal polypeptide domains (i), are characteristic of VDs of one or more antibodies directed against (i.e. recognizing, preferably specifically) an antigen present on the surface of mammalian cells, for example against the protein CD4, or against a viral antigen, in particular against a protein of a viral envelope, for example the external envelope protein of a HIV virus (human immunodeficiency virus), or against a bacterial antigen, in particular against a protein of a bacterial envelope, or against a retrotranscriptase, in particular a retrotranscriptase of HIV-1 or HIV-2.

A polypeptide domain (i) preferably consists of 10 to 300 amino acid residues, more preferably 20 to 200 or 40 to 200 amino acid residues (the lower and upper limits of these ranges are included).

In a particular embodiment of the invention, the nucleotide domains (ii) as defined in the present application are single stranded nucleic acids.

In a particular embodiment of the invention, the nucleotide domain (ii) of the chimeric molecule A and that of the chimeric molecule B of a half-antibody, which are single stranded structures, pair up in a hydric medium and in particular a reducing medium, into a double stranded structure.

In a particular embodiment of the invention, the nucleotide domains (ii) of A and B are selected from a polynucleotide, a PNA or a LNA and are capable of forming a stable double stranded structure, in particular an alpha helix.

In a particular embodiment of the invention, the sequences of the nucleotide domains (ii) of the two chimeric molecules A and B are complementary and antiparallel, and in particular are capable of hybridizing, preferably over their entire length. Hybridization of these two nucleotide domains means that a half-antibody molecule can be reconstituted, in particular in a hydric medium (including a reducing medium). Because of this hybridization, the peptide domains (i) of molecules A and B of a half-antibody assembled in this manner (said peptide domains if necessary being bonded to each other via a bond that is labile in a reducing medium, for example a disulfide bridge) are brought close together in space, which means that the epitope recognition site can be structured and stabilized, in particular in a reducing medium. Thus, even in a reducing medium, the antigen binding site is functional in the half-antibody of the invention.

In a particular embodiment of the invention, the size of domain (ii) varies from 3 to 100 nucleotides, more preferably 5 to 60 nucleotides (the lower and upper limits of these ranges being included).

In a particular embodiment of the invention,
the nucleotide domain (ii) of the chimeric molecule A or B of a half-antibody of the invention comprises or consists of the following sequence: ATGGTAGAG; and
the nucleotide domain (ii) of the other chimeric molecule of the half-antibody of the invention, respectively B or A, comprises or consists of the following sequence: CTCTACCAT.

In a particular embodiment of the invention,
the nucleotide domain (ii) of the chimeric molecule A or B of a half-antibody of the invention comprises or consists of the following sequence: CCAGCT; and
the nucleotide domain (ii) of the other chimeric molecule of the half-antibody of the invention, respectively B or A, comprises or consists of the following sequence: AGCTGG.

In a particular embodiment of the invention, a “linker” within the meaning of the present invention is a spacer, i.e. it can be used to separate the two domains it binds in space such that these two domains can exert their functions in an optimized manner.

In a particular embodiment of the invention, a “linker” within the meaning of the present invention preferably comprises 1 to 20 carbon atoms and comprises or consists of one or more element(s) (for example an element selected from a peptide (or a polypeptide), a polynucleotide, in particular a polynucleotide as defined in the present application (or an analog, in particular as defined in the present application), a polycarbon chain, ethylene diamine, polylysine, beta-alanine, and a sugar.

In a particular embodiment of the invention, said linker is a peptide or a polypeptide, which comprises or consists, for example, of one or more lysine(s). This linker will hereinafter be termed a “peptide linker”.

In a particular embodiment of the invention, a linker comprises or consists of a nucleic acid, in particular a DNA or a RNA and consists, for example, of a sequence of 5 to 50 nucleotides (the lower and upper limits of this range of values being included). This linker will hereinafter be termed the “nucleic linker”.

In a particular embodiment of the invention, the linker binding, as appropriate, the domains (i) and (ii) of a chimeric molecule A and the linker binding, as appropriate, the domains (i) and (ii) of a chimeric molecule B are peptide linkers.

In a particular embodiment of the invention, the domains (i) and (ii) are bonded via a linker (for example a peptide linker) in the two chimeric molecules A and B of a half-antibody, the linker present in A preferably being the same size as the linker present in B. As an example, the same linker could bind the domains (i) and (ii) in A and in B.

In a particular embodiment of the invention, a “linker” within the meaning of the present invention, for example a peptide or nucleic linker, comprises a bond that is labile in a reducing medium (for example a disulfide bridge) or comprises, at one of its ends, one or more amino acid(s) or nucleotide(s) that can be used to form such a bond with one of the two molecules it binds.

In a particular embodiment of the invention, “a bond that is labile in a reducing medium” means a bond that is labile in a reducing medium and persists in a non-reducing medium, said bond possibly being covalent (for example a disulfide bridge) or non-covalent. In accordance with a particular embodiment of the invention, in the chimeric molecule A, the C-terminal end of the polypeptide domain (i) (for example a V_{H} or V_{L} domain or a portion of a V_{H} or V_{L} domain) is covalently bonded to the 3′ end of a nucleic acid (ii) (for example a LNA, a methylphosphonate nucleic acid or a thioate nucleic acid) or to the C-terminal end of a PNA, for example via the NH₂ group of the side chain of a lysine bonded to the C-terminal end of said PNA via a peptide linkage.

Alternatively or cumulatively, in accordance with a particular embodiment of the chimeric molecule B, the C-terminal end of the polypeptide domain (i) (for example a V_{H} or V_{L} domain or a portion of a V_{H} or V_{L} domain) is covalently bonded to the 5′ end of the nucleic acid (ii) (for example a LNA, a methylphosphonate nucleic acid or a thioate nucleic acid) or to the N-terminal end of a PNA, for example via a peptide or amide linkage with one of the NH₂ groups of a lysine, said lysine being bonded to the N-terminal end of said PNA via its COOH group.

In a particular embodiment of the invention, the chimeric molecule A and/or the chimeric molecule B also comprises (comprise) a tag domain and/or a biologically active domain, said domain being bonded either to the free end of the nucleotide domain (ii) of the chimeric molecule A or B or to the free end of a linker bonded to the nucleotide domain (ii) of the chimeric molecule A or B. The bond of said tag domain or of said biologically active domain to the nucleotide domain (ii) of the chimeric molecule A or B or to the free end of said linker is preferably made via a bond that is labile in a reducing medium, for example a disulfide bridge, or in contrast via a bond that persists in a reducing medium, for example via a bond made with a maleimide acid.

In a particular embodiment of the invention, this linker is of sufficient length to pass through a membrane (for example a plasma membrane or an intracellular membrane).

In a particular embodiment of the invention, this linker is a nucleic linker.

Thus, in this particular embodiment, the half-antibody of the invention can be used to deliver, into a target cell (in particular a cell of a human or non-human mammal), a biologically active domain that can be used to inhibit (completely or partially) the expression of genes in said target cell, independently of the functional mechanisms intrinsic to said cell. In the particular embodiment using a bond that is labile in a reducing medium, the tag domain and/or the biologically active domain is liberated inside the cell after reduction of the disulfide bridges.

In a particular embodiment, the tag domain and/or the biologically active domain comprises or consists of a polynucleotide or an analog comprising or consisting of a tag sequence and/or a biologically active sequence that can be used to block (completely or partially) the expression of target genes (in particular as regards replication, transcription or translation), in particular in the interior of a cell, for example a polynucleotide or an analog selected from the following elements:
a nucleic acid, in particular an antisense nucleic acid or a nucleic acid with endonuclease activity, for example a ribonuclease (RNAse) or deoxyribonuclease (DNAse) activity;
a PNA, in particular an antisense PNA or a PNA with endonuclease activity (PNAzyme), for example a ribo- or deoxyribonuclease activity;
a LNA, a methylphosphonate nucleic acid, a nucleic acid methylsulfonate, a modified nucleic or deoxynucleic acid, a DNAzyme, a RNAzyme or a PNAzyme, a micro RNA (miRNA), a mixed PNA/nucleic acid or PNA/peptide sequence, a DNA/PNA-zyme dimer; and
a combination of at least two of said elements.

Alternatively or cumulatively, in a particular embodiment, the tag domain and/or the biologically active domain comprises or consists of a concatenation of a plurality (at least two) successive amino acid residues, forming the structure of a peptide or of a polypeptide. By way of example, said tag domain and/or said biologically active domain may comprise at least 4 or 6 amino acid residues, for example 4 to 200, 6 to 100 or 6 to 50 amino acid residues.

In the present application, a “peptide” denotes a chain of 2 to 20 successive residues (in particular 2, 3, 4, 5, 6, 7, 8, 9, 10, 11, 12, 13, 14, 15, 16, 17, 18, 19 or 20 residues), in particular a chain of 5 to 10, 10 to 15 or 15 to 20 successive amino acid residues.

In the context of the invention, a polypeptide, which also denotes a protein or a fragment of a protein, is a concatenation of more than 20 (at least 21) successive residues, in particular a chain of 21 to 1000 successive residues, preferably 21 to 500, 21 to 250 or 21 to 150 successive residues, and more preferably 21 to 50 or 21 to 100 successive amino acid residues.

Cumulatively or alternatively to the presence of a tag domain and/or biologically active domain in the chimeric molecule A and/or B, in accordance with a particular embodiment of the invention, the chimeric molecule A and/or the chimeric molecule B further comprises (comprise) a lipophilic (or hydrophobic) element, in particular a sequence, a chemical group or a lipophilic chemical compound, which preferably comprises one or more aromatic group(s) and/or compound(s), for example one or more tryptophan residues, said lipophilic element preferably being positioned at the free end of the chimeric molecule A or B. If appropriate, this lipophilic element is bonded to the chimeric molecule A or B via a bond that is labile in a reducing medium, for example a disulfide bridge.

In accordance with a particular embodiment of the invention, said or one of said lipophilic element(s) may be replaced by a beta-peptide domain, i.e. a domain consisting of or comprising one or more (for example 2, 3, 4, 5 or 6) beta-amino acid residues, for example 1 to 5 beta-amino acid residues. Said beta-peptide domain may, for example, comprise or consist of a chain of 2, 3, 4, 5 or 6 successive beta-amino acid residues. An example of a beta-amino acid that may be cited is beta-alanine.

In accordance with a particular embodiment, a half-antibody of the invention is such that:
the first chimeric molecule, A or B, comprises or consists of:
a polypeptide domain (i) as defined in the present application, and the following domains, positioned in succession in the following order, starting from the C-terminal end of the polypeptide domain (i):
if appropriate, a linker (for example a peptide linker) as defined in the present application;
a nucleotide domain (ii) as defined in the present application;
if appropriate, a linker (for example a nucleic linker), bonded, preferably via a bond that is labile in a reducing medium, for example a disulfide bridge or via a bond that persists in a reducing medium, for example via a bond established with a maleimide acid, to:
a tag domain or a biologically active domain as defined in the present application; and
if appropriate, a lipophilic element as defined in the present application, said lipophilic element if appropriate being bonded to the tag domain or to the biologically active domain via a bond that is labile in a reducing medium, for example a disulfide bridge, or via a linker (for example a peptide or nucleic linker) as defined in the present application; and
the second chimeric molecule, respectively B or A, comprises or consists of:
a polypeptide domain (i) as defined in the present application, this domain and the polypeptide domain (i) of the first chimeric molecule preferably being characteristic of VDs of the same antibody, one of these domains being characteristic of a VD of the light chain of a given antibody and the other being characteristic of a VD of the heavy chain of the same antibody;
   and the following domains, positioned in succession in the following order, starting from the C-terminal end of the polypeptide domain (i):
if appropriate, a linker (for example a peptide linker) as defined in the present application; and
a nucleotide domain (ii) as defined in the present application.

In a particular embodiment of the invention, the term “the following domains, positioned in succession in the following order” means “the following domains, bonded in succession in the following order”.

In a particular embodiment of the invention, a half-antibody of the invention, denoted HAB_{—}1, is such that:
the first chimeric molecule, A or B, comprises or consists of:
a polypeptide domain (i) as defined in the present application, and the following domains, positioned in succession in the following order, starting from the C-terminal end of the polypeptide domain (i):
if appropriate, a linker (for example a peptide linker) as defined in the present application;
a nucleotide domain (ii) as defined in the present application, termed S1;
if appropriate, a linker (for example a nucleic linker) as defined in the present application, bonded, preferably via a bond that is labile in a reducing medium, for example a disulfide bridge, to:
if appropriate, a tag domain or a biologically active domain as defined in the present application, bonded, if appropriate, for example, via a covalent bond, to:
if appropriate, a linker (for example a nucleic or peptide linker) as defined in the present application;
a second nucleotide domain termed S3, preferably bonded via a labile bond, which comprises or consists of a polynucleotide, in particular a DNA or a RNA, or an analog of a polynucleotide, in particular a PNA, a LNA, a methylphosphonate nucleic acid or a thioate nucleic acid, and which is capable of pairing into a double stranded structure with a complementary nucleotide domain termed S4, for example in a hydric medium, in particular in a reducing medium; and
if appropriate, a linker (for example a peptide linker) as defined in the present application;
the domain S3 or said linker being bonded to the C-terminal end of a second polypeptide domain, termed VD2a, which is characteristic of a VD of a heavy chain or of a light chain of an antibody, VD2a and the polypeptide domain (i) of this first chimeric molecule (A or B) preferably being characteristic of VDs of distinct antibodies; and
if appropriate, a lipophilic element as defined in the present application, said lipophilic element being bonded to VD2a via a bond that is labile in a reducing medium, for example a disulfide bridge, or via a linker (for example a nucleic linker) as defined in the present application, said linker comprising a bond that is labile in a reducing medium, for example a disulfide bridge or being bonded to VD2a via a bond that is labile in a reducing medium, for example a disulfide bridge; and
the second chimeric molecule, respectively B or A, comprises or consists of:
a polypeptide domain (i) as defined in the present application, this domain and the polypeptide domain (i) of the first chimeric molecule (of HAB_{—}1) preferably being characteristic of VDs of the same antibody, one of these domains being characteristic of a VD of the light chain of a given antibody and the other being characteristic of a VD of the heavy chain of the same antibody;
   and the following domains, positioned in succession in the following order, starting from the C-terminal end of the polypeptide domain (i):
if appropriate, a linker (for example a peptide linker) as defined in the present application; and
a nucleotide domain (ii) as defined in the present application, termed S2, capable of pairing into a double stranded structure with S1 in a hydric medium, in particular a reducing medium.

In a particular embodiment, a half-antibody of the invention, denoted HAB_{—}2, is such that:
the first chimeric molecule, A or B, comprises or consists of:
a polypeptide domain (i) as defined in the present application, and the following domains, positioned in succession in the following order, starting from the C-terminal end of the polypeptide domain (i):
if appropriate, a linker (for example a peptide linker) as defined in the present application;
the nucleotide domain (ii) as defined in the present application, termed S5;
if appropriate, a linker (for example a nucleic or peptide linker) as defined in the present application, said linker comprising, if appropriate, a bond that is labile in a reducing medium (for example a disulfide bridge), or being bonded via a bond that is labile in a reducing medium (for example a disulfide bridge) to:
a second nucleotide domain termed S4, preferably bonded via a labile bond, which comprises or consists of a polynucleotide, in particular a DNA or a RNA, or an analog of a polynucleotide, in particular a PNA, a LNA, a methylphosphonate nucleic acid or a thioate nucleic acid, and which is capable of pairing into a double stranded structure with a complementary nucleotide domain termed S3, for example in an aqueous medium, in particular in a reducing medium; and
if appropriate, a linker (for example a peptide linker) as defined in the present application;
the domain S4 or said linker being bonded to the C-terminal end of a second polypeptide domain, termed VD2b, which is characteristic of a VD of a heavy chain or of a light chain of an antibody, VD2b and the polypeptide domain (i) of this first chimeric molecule being characteristic of VDs of distinct antibodies; and
if appropriate, a lipophilic element as defined in the present application, said lipophilic element being bonded to VD2b via a bond that is labile in a reducing medium, for example a disulfide bridge, or via a linker (for example a nucleic linker) as defined in the present application, said linker comprising a bond that is labile in a reducing medium (for example a disulfide bridge) or being bonded to VD2a via a bond that is labile in a reducing medium (for example a disulfide bridge); and
the second chimeric molecule, respectively B or A, comprises or consists of:
a polypeptide domain (i) as defined in the present application, this domain and the polypeptide domain (i) of the first chimeric molecule (of HAB_{—}2) preferably being characteristic of VDs of the same antibody, one of these domains being characteristic of a VD of the light chain of a given antibody and the other being characteristic of a VD of the heavy chain of the same antibody;
   and the following domains, positioned in succession in the following order, starting from the C-terminal end of the polypeptide domain (i):
if appropriate, a linker (for example a peptide linker) as defined in the present application; and
a nucleotide domain (ii) as defined in the present application, termed S6, capable of pairing into a double stranded structure with S5 in a hydric medium, in particular a reducing medium.

In accordance with a particular embodiment, in HAB_{—}1 and in HAB_{—}2,
a bond that is labile in a reducing medium (for example a disulfide bridge) is present between domain (ii) and domain S3 (or S4); but
no bond that is labile in a reducing medium is present between domains S3 (or S4) and VD2a (or VD2b).

HAB_{—}2 may be used in conjunction with HAB_{—}1, for example administered to the same host, in particular to a human or non-human mammal, HAB_{—}1 and HAB_{—}2 being administered separately (HAB_{—}1 and HAB_{—}2 being present in distinct compositions, vesicles or viral particles of the invention, simultaneously or separately in time).

Different types of HAB_{—}1 and/or different types of HAB_{—}2 may be administered to the same host, in particular to a human or non-human mammal.

The HAB_{—}1 and HAB_{—}2 administered (simultaneously or separately in time) to the same host (in particular a human or non-human mammal), may also be administered in a form that is grafted via the bond that is labile in a reducing medium onto non-toxic nanoparticles, for example nanoparticles of diamond (see in particular the example of FIG. 10). HAB_{—}1 and HAB_{—}2 may in fact be bonded (or grafted) onto the same nanoparticle or onto two distinct nanoparticles. This grafting is accomplished via one or more bonds that are labile in a reducing medium, for example one or more disulfide bond(s), starting from the C-terminal end of the chimeric molecule (A or B) which comprises the domain VD2a or VD2b, and in particular (when VD2a or VD2b are in the C-terminal position in HAB_{—}1 or HAB_{—}2) starting from the N-terminal end of the VD2a or VD2b portion of HAB_{—}1 or of HAB_{—}2, if appropriate via a linker.

Hence, the systems S3-VD2a and S4/VD2b may be delivered (via HAB_{—}1 and HAB_{—}2 respectively) to the same cell when HAB_{—}1 and HAB_{—}2 or the nanoparticle(s) onto which HAB_{—}1 and/or HAB_{—}2 are grafted are internalized and the labile bonds (for example the disulfide bridges) are cleaved.

The half-antibodies HAB_{—}1 and/or HAB_{—}2 grafted onto the same nanoparticle may be identical or different, and in particular may differ in their S3/VD2a (or S4-VD2b) domains and/or their (i)/(ii) domains. Hence, the HAB_{—}1 and/or HAB_{—}2 grafted onto the same nanoparticle may comprise:
a single type of S3/VD2a (or S4-VD2b) domains and a single type of (i)/(ii) domains;
   different types of S3/VD2a (or S4-VD2b) domains and a single type of (i)/(ii) domains;
a single type of S3/VD2a (or S4-VD2b) domains and different types of (i)/(ii) domains; or
different types of S3/VD2a (or S4-VD2b) domains and different types of (i)/(ii) domains.

Domains said to be “x/y” are said to be “of different types” in the context of the present application when they differ in their x domain and/or in their y domain. In one embodiment, both x and y differ between two x/y domains “of different types”.

A nanoparticle onto which HAB_{—}1 and/or HAB_{—}2 are grafted comprising different types of (i)/(ii) domains can be used to target different epitopes and thus can generally be used to target different types of cells. In contrast, a nanoparticle onto which HAB_{—}1 and/or HAB_{—}2 are grafted comprising a single type of (i)/(ii) domains can be used to target a single type of epitope and thus generally a single type of cells.

In a particular embodiment, the nanoparticles used have a mean size in the range 1 to 100 nanometers, preferably in the range 1 to 50 nanometers. As an example, their mean size may be 10 nanometers or less than 10 nanometers.

Thus, in host cells that have integrated both HAB_{—}1 and HAB_{—}2, a third type of half-antibody, constituted by a part of HAB_{—}1 and by a part of HAB_{—}2, may be formed.

By way of example, in a particular embodiment, this half-antibody, denoted HAB_{—}3, is such that:
the first chimeric molecule, A or B, comprises or consists of:
the polypeptide domain VD2a, and the following domains, positioned in succession in the following order, starting from the C-terminal end of the domain VD2a:
if appropriate, a linker (for example a peptide linker) as defined in the present application;
the nucleotide domain S3;
if appropriate, a linker (for example a nucleic or peptide linker) as defined in the present application; and
the tag domain or the biologically active domain present in HAB_{—}1; and
the second chimeric molecule, respectively B or A, comprises or consists of:
the polypeptide domain VD2b, and the following domains, positioned in succession in the following order, starting from the C-terminal end of the domain VD2b:
if appropriate, a linker (for example a peptide linker) as defined in the present application; and
the nucleotide domain S4.

In a particular embodiment of the invention, one of the domains VD2a and VD2b as defined in the present application is characteristic of a VD of the light chain of a given antibody and the other of these domains is characteristic of a VD of the heavy chain of the same antibody.

When half-antibodies HAB_{—}1 and half-antibodies HAB_{—}2 are used and in particular when they are present on the same particle (in particular the same nanoparticle), it may be advantageous to protect (temporarily) the portions S3 and S4, for example by hybridizing these domains with a RNA. This can then (temporarily) prevent any possible association between S3 and S4 so that these two domains cannot become associated before having reached the target cell (in particular it can avoid a premature association of S3 and S4 when HAB_{—}1 and HAB_{—}2 are grafted onto the same particle). The RNAs are then dehybridized or digested, which then makes an association between S3 and S4 possible.

When an antigen of interest that is soluble, in particular circulating in a host (i.e. it is not expressed on the surface of a cell) is to be targeted, it may be advantageous to use one (or more) half-antibodies HAB_{—}1 in conjunction with one (or more) half-antibodies HAB_{—}2, and to design these half-antibodies so that the paratope of HAB_{—}1 and the paratope of HAB_{—}2 (i.e. the recognition site present at their N-terminal end) are specific for the same soluble antigen or, more specifically, for the same epitope of this soluble antigen.

In accordance with a particular embodiment, a half-antibody HAB_{—}1 and a half-antibody HAB_{—}2 are associated so as to reconstitute a complete antibody (having two epitope recognition sites). Such reconstituted antibodies may in particular be used for therapeutic and/or diagnostic purposes, in particular when the antigen(s) of interest is (are) soluble. By way of example, it is possible to use HAB_{—}1s and HAB_{—}2s wherein the paratopes are specific for the same soluble antigen or, more specifically, the same epitope of this soluble antigen.

The invention also concerns a chimeric molecule A or B as defined in the present application.

The invention also concerns a vesicle, in particular a liposome, and more particularly a liposome constituted by a viral membrane, for example a membrane of particles of the HIV-1 or HIV-2 virus, or a viral particle, said vesicle or said viral particle comprising one or more half-antibodies as defined in the present application and/or one or more chimeric molecule(s) A and/or B as defined in the present application.

The invention also concerns nanoparticles (in particular nanoparticles as defined in the present application) onto which one or more half-antibodies as defined in the present application and in particular one or more half-antibodies HAB_{—}1 and/or one or more half-antibodies HAB_{—}2 and/or one or more half-antibodies HAB_{—}4 (see below) are bonded (or grafted) via bonds that are labile in a reducing medium, for example a disulfide bridge (see FIG. 11).

By way of example, in a particular embodiment, the vesicle or said viral particle, the particle or the nanoparticle as defined in the present application comprises a half-antibody HAB_{—}1 or a half-antibody HAB_{—}2 as defined above, and in particular:
a half-antibody HAB_{—}1, in which:
the polypeptide domain (i) of the chimeric molecules A and B is preferably characteristic of the same antibody directed against a first antigen present on the surface of mammalian cells or directed against a viral antigen, for example against the external envelope protein of a HIV virus; and
the polypeptide domain VD2a is preferably characteristic of an anti-retrotranscriptase antibody, in particular anti-retrotranscriptase of HIV-1 or HIV-2; or
a half-antibody HAB_{—}2, in which
the polypeptide domain (i) of the chimeric molecules A and B is preferably characteristic of a VD of the same antibody directed against a second antigen present on the surface of mammalian cells, for example against the protein CD4, or directed against a viral antigen; and
the polypeptide domain VD2b is preferably characteristic of a VD of the same antibody as the VD domain VD2a, VD2a or VD2b being characteristic of a VD of the light chain of a given antibody, for example an anti-retrotranscriptase antibody, and the other domain, respectively VD2b or VD2a, being characteristic of a VD of a heavy chain of the same antibody.

In a particular embodiment of the invention, two half-antibodies are used, for example a half-antibody HAB_{—}1 and a half-antibody HAB_{—}2 as defined in the present application. One of these half-antibodies, for example HAB_{—}2, may be present in a form that is incorporated into a vesicle, a viral particle or fixed on one or more particle(s) or nanoparticle(s) as defined in the present application, the other half-antibody, for example HAB_{—}1, being present in solution. HAB_{—}1 and HAB_{—}2 may in particular be respectively directed against an epitope of a HIV-1 or HIV-2 and an epitope expressed at the surface of mammalian cells, in particular T cells, for example an epitope of the CD4 protein of CD4⁺cells.

The invention also envisages a composition, in particular a pharmaceutical or therapeutic, immunological or vaccine composition comprising, consisting or essentially consisting of:
one or more half-antibodies as defined in the present application and/or one or more chimeric molecule(s) A and/or B as defined in the present application, or one or more vesicle(s) or viral particle(s) of the invention, or one or more particle(s) or nanoparticle(s) as defined in the present application; and
if appropriate, a support, a diluent and/or a pharmaceutically acceptable vehicle.

The expression “essentially consists of” means that in addition to the expressly listed elements, other minor ingredients or molecules may be present in the composition of the invention without in any way affecting the activity of the expressly listed elements.

A substance or a combination of substances is said to be “pharmaceutically acceptable” when it is appropriate for administration to a host for therapeutic or prophylactic purposes. It is thus preferably non-toxic for the host to which it is administered.

A “host” in the context of the present invention in particular denotes a human host (or patient) or a non-human animal, for example a human or non-human mammal

In a particular embodiment, the composition of the invention comprises, consists or essentially consists of the half-antibody HAB_{—}1 and/or the half-antibody HAB_{—}2 as defined in the present application.

The invention also concerns a kit comprising:
one or more element(s) selected from: a half-antibody of the invention, a chimeric molecule A or B of the invention, a vesicle of the invention, a viral particle of the invention, a particle or nanoparticle as defined in the present application and a composition of the invention;
and if appropriate, instructions for use.

In a particular embodiment, the kit of the invention comprises the half-antibody HAB_{—}1 and/or the half-antibody HAB_{—}2 as defined in the present application.

The invention also provides one or more element(s) selected from: a half-antibody of the invention, a chimeric molecule A or B of the invention, a vesicle of the invention, or a particle or nanoparticle as defined in the present application, a viral particle of the invention, a composition of the invention or a kit of the invention, for use as a drug. Said elements may in particular be used in a host, for example a human or a non-human mammal, for the prevention and/or treatment of a genetic disease, for example a myopathy, and/or an infectious disease, in particular a viral disease and more particularly an infection by a lentivirus, for example HIV-1 or HIV-2, and/or a bacterial infection, for example an infection by a bacterium that is resistant to antibiotics and/or for the prevention or the treatment of a cancer.

The invention also concerns the use of one or more elements selected from those indicated above, for the manufacture of a drug intended to prevent or treat the diseases or infections mentioned above, in a host, for example a human or non-human mammal

The invention also concerns a method for removing or treating the diseases or infections mentioned above in a host requiring it, said method comprising or consisting of a step for administering one or more elements selected from those indicated above to said host.

In order to enhance the beneficial effects of these elements, administration may be carried out in the form of a plurality of administrations in succession, repeated on one or more occasions, after a particular time interval.

The invention also concerns the use of one or more elements selected from those indicated above, to tag and/or amplify protein tagging, for example on the surface of a cell, in vitro, in vivo and ex vivo.

The invention also concerns a method for synthesizing a half-antibody as defined in the present application. This method may comprise a step in which grafting or bonding onto the C-terminal end of the polypeptide domain (i) (for example the C-terminal end of a V_{H} or V_{L} domain), of a nucleotide domain (ii), for example a PNA modified at the N- or C-terminal end by a lysine, or a nucleic acid modified at the 5′ or 3′ end by a NH₂, is carried out by the action of 2-mercapto-ethanesulfonic acid or 3-mercaptopropionic acid on a cysteine of said polypeptide domain (i). The lysine or modified nucleic acid may be replaced by a beta-alanine, an ethyl diamine, a NH—CH(CONH₂)—(CH₂)₄NH, or a NH₂(CH₂)₄(NH₂)CH(COOH).

Alternatively or cumulatively, the method for synthesizing a half-antibody of the invention may comprise a step in which the polypeptide domains (i) of said half-antibody, which are VDs of antibodies (for example the V_{H} or V_{L} domains of antibodies or comprising one or more portion(s) of a V_{H} or V_{L} domain of the antibody) are obtained either by digestion of one or more antibodies by an enzyme, for example pepsin, papain, trypsin, or by the expression of polypeptides coding for these VDs, in systems such as bacteria, yeasts, insect cells, mammalian cells, or by in vitro syntheses, then by purification.

Alternatively or cumulatively, in order to clone the polypeptide domains, for example VDs as defined in the present application, these domains (or their nucleic sequence to be cloned) are fused at their C-terminal end (respectively 3′ of the sequence to be cloned) with a peptide sequence LPXTAAAA (leucine-proline-X-threonine-(alanine)₄, where X is any amino acid) respectively the nucleic sequence coding for LPXTAAAA. The nucleotide domain to be grafted, in particular the nucleic acid or the PNA to be grafted, may itself be functionalized at one of its ends by a poly-G (poly-glycine), optionally via a lysine, the N-terminal end of the poly G remaining free. A polypeptide domain (i) (for example a VD as defined in the present application) and a nucleotide domain (ii) (for example a nucleic acid (or polynucleotide) or a PNA as defined in the present application) are then fused under the action of a sortase A.

The sequence LPXTAAAA may advantageously be replaced by the sequence LPXTG (leucine-proline-X-threonine-glycine, where X is any amino acid; SEQ ID NO. 7).

Alternatively or cumulatively, fusion between the VD and the PNA may be obtained by di-methylSiCl₂ (dimethyldichlorosilane) catalysis, allowing an amine function to be grafted at the C-terminal end of a peptide.

The invention also pertains to the amplification capacity of the sequence by PCR at the tag of a chimera once it has recognized an AG fixed on a support that can be used to identify and quantify the AG even if present in a single copy.

The invention also pertains to a method for detecting and, if appropriate, quantifying one or more antigen(s) of interest (more generally one or more proteins of interest) possibly present in a sample, in particular a biological sample, said method comprising or consisting of the following steps:
bringing the antigens of the sample into contact with one or more half-antibodies as defined in the present application, in particular with the half-antibodies HAB_{—}1 and/or HAB_{—}2;
detecting, for example by PCR, any complexes formed between said half-antibody or antibodies and one or more antigen(s) of the sample;
if appropriate, quantifying the antigen or antigens detected thereby.

A complex between a half-antibody and an antigen of interest of the sample is formed by recognition of an epitope of the antigen of interest by the half-antibody recognition site.

In a particular embodiment of the invention, the antigens (or more generally the proteins) that may be present in the biological sample have been fixed on a solid support, a particle or any other element, allowing a complex formed between a half-antibody being used and an antigen of the sample from the remainder of the sample being analyzed. Alternatively, the method may include a first step consisting of fixing antigens (or more generally proteins) that may be present in the biological sample on a solid support, a particle or any other element allowing said complex to be isolated.

In a particular embodiment of the invention, detection and, if appropriate, quantification of antigens present in the sample are carried out by detecting a particular sequence present in the half-antibody employed. In particular, detection and, if appropriate, quantification of antigens present in the sample may be carried out by amplifying, for example by PCR, a particular nucleotide sequence present in the half-antibody or one of the half-antibodies employed (for example a particular nucleotide sequence present in a tag domain of said half-antibody). Hence, provided that a sufficient quantity of half-antibody is used and that the half-antibodies that have not hybridized have been eliminated before carrying out the amplification, the recorded amplification will be proportional to the number of molecules of half-antibodies complexed to a given antigen (and as a result proportional to the number of molecules of a given antigen).

The term “biological sample” as used in the present application means a sample of blood, plasma, serum, urine or any other fluid that is capable of being obtained from a human or non-human host (in particular a mammal), or a tissue or cell sample (or even a single cell).

The invention also pertains to the use of one (or more) half-antibodies of the invention for carrying out an analysis method using a nucleic acid chip (or biochip) (in particular with DNA and/or RNA and/or PNA and/or LNA, etc.) to produce an antibody chip or a mixed antibody/nucleic acid chip.

Thus, the invention also pertains to a second method for detecting and, if appropriate, quantifying one or more antigen(s) of interest (more generally one or more proteins of interest) that may be present in a sample (in particular a biological sample), said method comprising or consisting of the following steps:

a) bringing one or more hybridization units (or spots) of a nucleic acid chip into contact with one (or more) half-antibodies as defined in the present application and in particular with a half-antibody HAB_{—}1, HAB_{—}2, or HAB_{—}4, the half-antibody being capable of becoming attached (in particular of hybridizing) to one or more hybridization unit(s) of said chip;

b) bringing hybridization units of the chip (or at least the hybridization units that have been brought into contact with the half-antibody in step a)) into contact with the proteins or antigens of the sample (or more generally with the sample);

c) detecting complexes that may be formed between the half-antibody attached to the chip at the end of step b) and one or more antigen(s) of the sample;

d) if appropriate, quantifying the antigen(s) detected thereby.

In a particular embodiment of the invention, step a) is dispensed with in the method described above, the chip employed in the context of this method being an antibody chip or a mixed antibody/nucleic acid chip as defined below.

In a particular embodiment of the invention, said method for analysis by biochip comprises or consists of the following steps:

a) step a) as defined above;

b) bringing the hybridization units of the chip (or at least the hybridization units that have been brought into contact with the half-antibody in step a)) into contact with the sample);

c) detecting complexes that may be formed between the half-antibody fixed on the chip at the end of step b) and one or more antigen(s) of the sample;

d) detecting complexes that may be formed between, on the one hand, nucleic acids or PNAs present on one or more hybridization unit(s) of the chip and on the other hand the complementary nucleic acids present in the sample;

e) if appropriate, quantifying the antigen or antigens of the sample detected in step c) and/or the nucleic acid(s) of the sample detected in step d).

Step d) may be carried out before or after step c) or simultaneously with step c). In a particular embodiment of the invention, the steps of detection and/or quantification, or certain detection and/or quantification steps, are carried out by PCR amplification and/or by confocal microscopy.

In a particular embodiment of the invention, steps a) and b) are replaced by the following steps:

a) one (or more) half-antibodies as defined in the present application, in particular a half-antibody HAB_{—}1, HAB_{—}2 or HAB_{—}4, is brought into contact with the proteins or antigens of the sample (or more generally with the sample) under conditions that allow the formation of protein/HAB complexes; then

b) the half-antibody of step a) is brought into contact with one or more hybridization units (or spots) of a nucleic acid chip to which the half-antibody is capable of attaching (in particular by hybridizing)

In a particular embodiment, in step b), the half-antibodies brought into contact with the hybridization units are half-antibodies present in the form of protein/HAB complexes.

In a particular embodiment, the chips used in the context of the invention are impedance chips, in particular chips as disclosed in applications WO 2005/036170 and WO 2006/090073.

In a particular embodiment of the invention, the half-antibody or half-antibodies or one of the half-antibodies used in the biochip analysis method of the invention is (are) such that it (they) comprise(s) a nucleotide domain (for example a tag domain) that is complementary to a nucleotide sequence present on one or more hybridization unit(s) of the chip used (see FIG. 12).

By way of example, in a particular embodiment of the invention, the half-antibody used in the biochip analysis method of the invention is a half-antibody HAB_{—}4, wherein:
the first chimeric molecule, A or B, of this half-antibody comprises or consists of:
a polypeptide domain (i) as defined in the present application, and the following domains, positioned in succession in the following order, starting from the C-terminal end of polypeptide domain (i):
if appropriate, a linker (for example a peptide linker) as defined in the present application;
a nucleotide domain (ii) as defined in the present application;
if appropriate, a linker (for example a nucleic linker), bonded, preferably via a bond that is labile in a reducing medium (for example a disulfide bridge) or via a bond that persists in a reducing medium (for example via a bond established with a maleimide acid), to a tag domain that is complementary to a nucleotide sequence of a hybridization unit on said chip (which allows binding of the chimeric molecule to the specific hybridization unit of the chip, thereby transforming the hybridization unit into a complexing unit for attachment to a target by antibody-antigen recognition; and
second chimeric molecule, respectively B or A, of this half-antibody comprises or consists of:
a polypeptide domain (i) as defined in the present application, this domain and the polypeptide domain (i) of the first chimeric molecule preferably being characteristic of VDs of the same antibody, one of these domains being characteristic of a VD of the light chain of a given antibody and the other being characteristic of a VD of the heavy chain of the same antibody;
   and the following domains, positioned in succession in the following order, starting from the C-terminal end of the polypeptide domain (i):
if appropriate, a linker (for example a peptide linker) as defined in the present application; and
a nucleotide domain (ii) as defined in the present application, capable of pairing in a hydric medium (in particular a reducing medium) into a double stranded structure with the nucleotide domain (ii) of the first chimeric molecule, respectively A or B.

The invention also concerns a chip (or biochip) as defined in the present application, which comprises one or more hybridization units on which one (or more) half-antibodies as defined in the present application is complexed (or bound). Said chip is thus denoted an “antibody chip (or biochip)” (when all of the hybridization units of said chip are hybridized with a half-antibody) or a “mixed antibody/nucleic acid chip” (when one or more hybridization units of said chip are not hybridized with a half-antibody but with a nucleic acid).

The invention also provides a kit, in particular a kit that is suitable for use in detecting and, if appropriate, quantifying one or more antigen(s) of interest that may be present in a sample, said kit comprising or consisting of:

a)—a nucleic acid chip; and
one or more half-antibodies as defined in the present application or one or more composition(s) comprising one or more half-antibodies of the invention; and
if appropriate, instructions for use; or

b)—an antibody chip or a mixed antibody/nucleic acid chip of the invention; and
if appropriate, instructions for use.

Thus, the present invention can be used to functionalize complexing units of a nucleic acid chip so as to provide said complexing units with the ability to recognize antigens and in particular epitopes.

In similar manner, it is possible to functionalize any other element presenting a surface, in particular particles, by the half-antibodies of the invention, for example via PNA or nucleic acids that are complementary to a tag domain present in said half-antibodies (see FIG. 13).

Thus, the invention also pertains to a particle (for example a nanoparticle), in particular a fluorescent or non-fluorescent particle, characterized in that one or more half-antibodies as defined in the present application, in particular one or more half-antibodies HAB_{—}1 and/or one or more half-antibodies HAB_{—}4, are bonded to the surface of said particle. The half-antibody/particle bond may be carried out via a PNA or a nucleic acid comprising a sequence that is complementary to a nucleic sequence of said half-antibody (for example complementary to the tag domain of the half-antibody).

Said particle may, for example, be a quantum dot (or Q-dot).

Said particle may in particular be used to tag (in vivo, ex vivo or in vitro) target cells, i.e. cells on the surface of which an epitope is present that is recognized by the recognition site of a half-antibody of the invention.

The invention also concerns addressing (in vivo, ex vivo or in vitro) one or more (in particular 2) PNAzymes to target cells.

The invention in fact concerns a catalytic PNA (denoted PNAzymes in the present application) that has the capacity of specifically cleaving a DNA. This PNAzyme comprises or consists of one or more HD (histidine-aspartic acid) or DH (aspartic acid-histidine), EH (glutamic acid-histidine) or HE (histidine-glutamic acid) motif(s). The use of a single PNAzyme means that the DNA can be cleaved, while using two PNAzymes such that two contiguous, non-complementary sequences located on the two strands of a complementary DNA are cleaved can be used to cleave the two strands in the manner of a restriction enzyme, producing an offset double stranded cut; using a contiguous PNAzyme system means that deletions can be introduced into the DNA.

The PNAzyme system or systems may be supplied to a target cell by any means, in particular using one or more half-antibodies of the invention.

Thus, the invention in particular concerns a half-antibody of the invention in which the tag domain and/or the biologically active domain comprises or consists of one or more PNAzymes (in particular one or more PNAzymes comprising a HD or DH motif). When two PNAzymes are used in the context of the invention, they are preferably carried by the same half-antibody, which can be grafted onto a non-toxic nanoparticle (as described in the present application).

In a particular embodiment, the invention concerns a half-antibody as defined in the present application, in which the tag domain and/or the biologically active domain comprises or consists of two PNAzymes, in particular two PNAzymes with a HD or DH motif, which are such that the two PNAzymes are bonded by their N-terminal ends or possibly via their C-terminal ends via the same linker (these two PNAzymes are denoted herein “head-to-tail double PNAzymes” or “htDAZ” such that these PNAzymes recognize two non-complementary contiguous sequences located on the two strands of a complementary DNA (see FIG. 14). By hybridizing to DNA, htDAZ can be used to produce a double stranded offset cleavage between the two strands of a DNA molecule. Addressing the two htDAZs by two chimeric molecules fixed, for example, to the same nanoparticle, and comprising the same VD domain but two different htDAZs (thus defining the offset cleavage sites), frames a given region of a DNA molecule, meaning that deletion of this region can be accomplished.

This tool can in particular be used to delete the region of the nucleotide sequence of a virus, for example a HIV virus, in particular a HIV-1 virus, providing resistance to said virus (see FIG. 15), or to delete a region of the nucleotide sequence of a bacterium providing resistance to one or more antibiotic(s); by way of example, the sequence PBS or sequences responsible for penetration into the nucleus may be cited.

The invention also concerns an enzyme or PNAzyme per se, formed by a structure comprising or consisting of:
a PNA sequence, bonded to
a peptide sequence, bonded to
another PNA sequence;
   the two PNA sequences possibly being identical or different.

In a particular embodiment of the invention, the PNA sequences are determined in order to target a particular nucleic acid sequence (for example a particular DNA sequence) and to pair with it.

In particular, the invention concerns two PNAzyme structures as defined above (these two PNAzymes possibly having identical or different sequences) bonded so that they are head-to-tail, i.e. bonded via their C-terminal ends or (preferably) via their N-terminal ends, preferably via the same linker. Such a double PNAzyme structure is termed a “head-to-tail double PNAzyme”.

The term “peptide sequence” as used here means a chain of at least two (i.e. two or more than two) amino acids.

In a particular embodiment of the invention, the peptide sequence present in a PNAzyme constitutes a catalytic domain of an enzyme, in particular a nuclease, preferably a DNAse.

In a particular embodiment of the invention, said peptide sequence comprises or consists of one or more DH (aspartic acid-histidine), HD (histidine-aspartic acid), EH (glutamic acid-histidine) or HE (histidine-glutamic acid) motifs.

A PNAzyme, in particular a head-to-tail double PNAzyme, can be used to carry out, as with a restriction enzyme, single strand or double strand cleavage in a DNA. In addition, using two head-to-tail double PNAzymes (i.e. 4 PNAzymes) can create deletions in a double stranded DNA, if appropriate using adjacent cuts on two complementary DNA strands.

Other particular embodiments, characteristics and advantages of the invention will become apparent from the following examples as well as the figures, in particular in FIG. 12.

### DESCRIPTION OF THE FIGURES

FIG. 1: Example of synthesis. 1: native antibody; 2: disulfide bridge (or S-S bridge); 3: light chain and heavy chain variable domains (VD); 4: structural nucleotide domains (structural PNA or zipper); 5. half-antibody; 5a: active site (VD1a+VD1b; site comprising an epitope recognition site) of structured half-antibody; 5b: paired structural nucleic acids forming a zipper type structure; 6-8: tag nucleotide sequence comprising a biologically active sequence; 6: linker/spacer sequence; 7: labile disulfide bridge; 8: biologically active sequence (PNA, LNA . . . ); 9: lipophilic element (for example: tryptophan).

FIG. 2. Reactor. 10: electrode or electrode and gel connected to terminals of a generator; 11: brass tube containing the solution to be reduced; 12: duracryl gel & SH beads; 13: Electrically insulating plastic tube (Tycon); 14: Electrode guide; 15: Cooling coil, radiator in which water circulates at a temperature of less than 10° C.

FIG. 3. 16: Western blot analysis of native antibodies; 17: Western blot analysis of denatured antibodies; 18: Western blot analysis of antibodies electro-reduced in the brass/Tycon reactor at 100V, 2 mA, 10 sec.

FIG. 4. 19: antisense locking nucleic acids; 22: Interaction, detected by NMR, between a PNA w-GTCCCGUCCCCGCCA-c; SEQ ID No. 1 (w: tryptophan; c: cysteine); 23: Loop of RNAse or DNAse activity of a PNA/DNAzyme DNAzyme; 24: DH or HD motif of an enzymatic loop of a PNAzyme.

FIG. 5. 6: linker/spacer sequence; 7: labile disulfide bridge; 8: biologically active sequence (PNA, LNA . . . ); 25: HAB_tag (1); 26: structural PNA 1 (S3); 27: VD2a); 28: VD2b); 29: structural PNA 1 (S4); 30: HAB_tag (2); 31: HAB_tag (3).

FIG. 6: 20: interaction, detected by NMR, between a PNA w-CGCCA-c (w: tryptophan and c: cysteine with N-terminal 5′ and C-terminal 3′); 21:Interaction, NMR analysis, between a PNA w-GTCCC-c (w: tryptophan and c: cysteine with N-terminal 5′ and C-terminal 3′); 22: interaction, detected by NMR, between a PNA w-GTCCCGUCCCCGCCA-c; SEQ ID No. 1 (w: tryptophan; c: cysteine).

FIG. 7: antibody/antigen recognition on nitrocellulose membrane. A. electro-reduced antibodies+BSA. B. chimera+BSA.

FIG. 8: results of fluorescence tagging on nitrocellulose membrane. (1) Fluorescence of chimera/BSA supernatant on nitrocellulose; (2) negative control (fluorescent probe on BSA).

FIG. 9: results of fluorescence on magnetic traps. (1) supernatant from magnetic traps/tagged chimeras; (2) negative control (fluorescent probe with magnetic traps).

FIG. 10: free chimera and chimera grafted onto particles. 7: labile disulfide bridge; 25: HAB_tag (1); 26: structural PNA 1 (S3); 27: VD2a); 28: VD2b); 29: structural PNA 1 (S4); 30: HAB_tag (2); 31: HAB_tag (3); 32: particle.

FIG. 11: chimera grafted onto particles. 7: labile disulfide bridge; 25: HAB_tag (1); 26: structural PNA 1 (S3); 27: VD2a); 28: VD2b); 29: structural PNA 1 (S4); 30: HAB_tag (2); 31: HAB_tag (3); 32: particle.

FIG. 12: mixed protein/nucleic acid nucleic acid chips. 8′: tag sequence (PNA, LNA . . . ); 25: HAB_tag; 35: chip (PNA, LNA, nucleic acids . . . ); 34 PNA anti-tag sequence spot; 36: PNA anti-DNA/RNA sequence spot. 37: target nucleic acid.

FIG. 13: fluorescent particle functionalization. 8′: tag sequence (PNA, LNA . . . ); 25: HAB_tag; 38: fluorescent particle; 39 complementary sequence of tag.

FIG. 14. 24: DH or HD of a PNAzyme enzymatic motif; 40: PNA portion of PNAzyme; 41: NN or CC linker cleavage site between complementary PNA, LNA . . . ; 42: linker binding C-terminal portions of two head-to-tail PNAzymes; 43: DNA target to be deleted.

FIG. 16. 6: linker/spacer sequence; 44: structural PNA S1; 45: DVA; 46: DVB; 47: structural PNA (S2).

FIG. 17: Results of dotblots on the purification of molecules SP1 & SP2.

48: Supernatant 1/SP(1/2) molecules→solution comprising the proteins HS-LPGTG+the enzyme sortase A+PNA SP(1/2); 49: enz AcTev/SP(1/2) Molecules elution_→solution comprising the proteins HS-LPGTG+the enzyme sortase A+PNA SP(1/2); 50: imidazole/SP(1/2) Molecules elution→solution comprising the proteins HS-LPGTG+the enzyme sortase A+PNA SP(1/2);
51: Supernatant 1/CNI→negative control 1, solution comprising the proteins HS-LPGTG+the enzyme sortase A; 52: enz AcTev/CNI enzyme→negative control 1, solution comprising the proteins HS-LPGTG+the enzyme sortase A; 53: imidazole/CNI elution→negative control 1, solution comprising the proteins HS-LPGTG+the enzyme sortase A;
54: Supernatant 1/CNII→negative control II, solution comprising the proteins HS-LPGTG+PNA SP1; 55: enz AcTev/CNII elution→negative control II, solution comprising the proteins HS-LPGTG+PNA SP1; 56: imidazole/CNII elution→negative control II, solution comprising the proteins HS-LPGTG+PNA SP1;
Supernatant 1: protein solution not retained on NI-NTA beads.
enz Actev elution: first elution of proteins retained on NI-NTA beads by the enzyme AcTev. Imidazole elution: second elution of proteins retained on NI-NTA beads by 400 mM imidazole.

FIG. 18: Dotblotting antigen recognition (HEL_DL488)/zipped antibody. 56: native anti-HEL AB undiluted, deposited on nitrocellulose (1×2 uL to 650 uM)+HEL_DL488 in solution; 57: native anti-HEL AB, diluted (5*×2 uL, approximately 10 uM)+HEL_DL488 in solution; 58: paired SP1&SP2 molecules (variable portion of zipped antibody)(5×2 uL, approximately 10 uM)+HEL_DL488 in solution.

FIG. 19: Result of digestion of PBS from HIV-I by the PNAzyme PZ1; 59: uncut PBS RNA; 60: cleaved PBS RNA; 61: 20% acrylamide native gel; 62: 20% acrylamide denaturing gel; 63: PBS/PNAZYME duplex; 64: PBS alone; 65: PBS+PZ1 at T0; 66 PBS+PZ1 at T6h, disappearance of PBS; Bp: molecular weight marker; A: PBS (RNA) alone; B: PBS (RNA)+PZ1 (6 h at 37° C.); C: PBS (RNA)+PZ1 (Oh at 37° C.); D: PBS (RNA)+PZ1 (6 h at 37° C.).

### EXAMPLES

Novel chimeric molecules known as “half-antibodies”, which are capable of self-assembling in a suitable medium, were synthesized. They comprise a portion with a polypeptide nature and a portion constituted by a nucleotide sequence or an analog.

Preferably, the polypeptide portion is constituted by antibody portions, and more precisely variable domains of one or more antibodies that are obtained from the heavy and light chains of an antibody and which, by becoming structured, specifically form an epitope recognition site. The second portion is constituted by a nucleic acid or an analog such as a PNA or a LNA.

A first type of HAB consists of two VD domains (one domain obtained from a heavy chain of an antibody and one domain obtained from a light chain of the same antibody, which specifically recognize the antigen) and of two nucleic sequences.

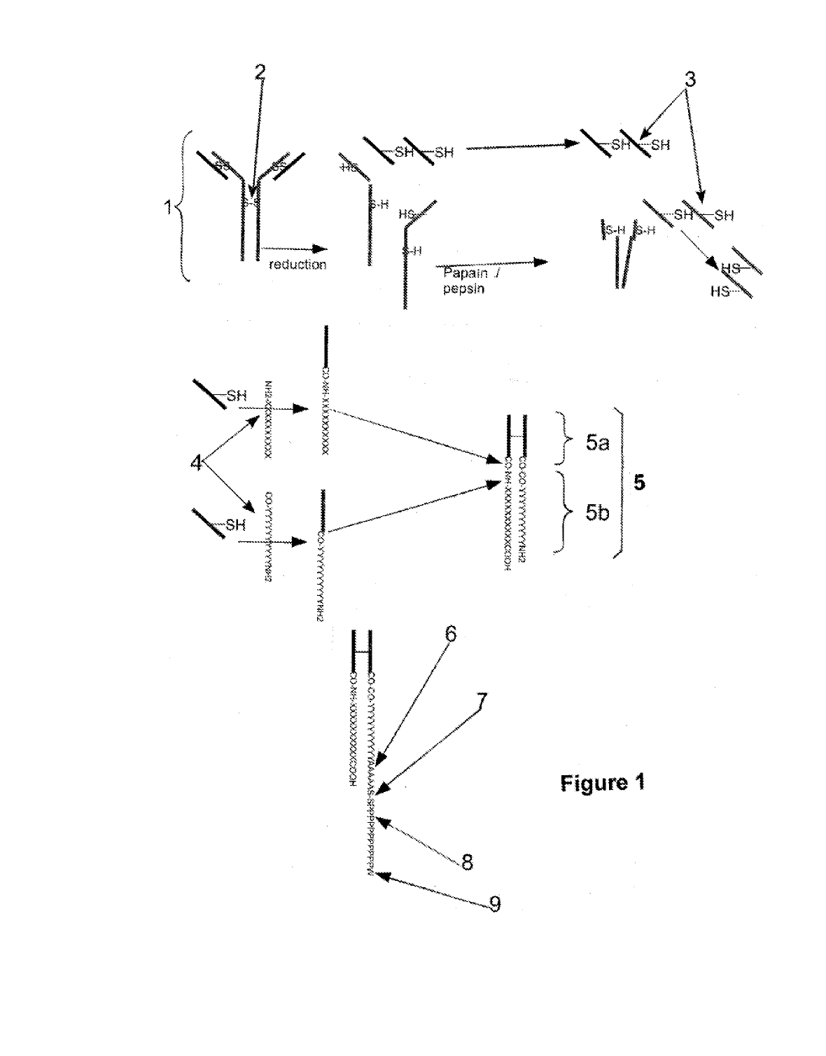

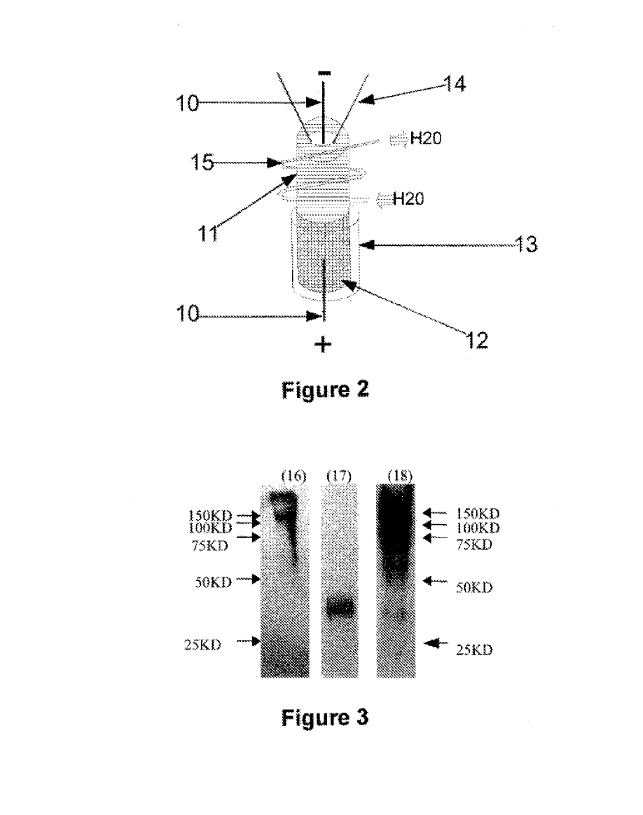

One of the VD domains (FIG. 1.3) is covalently coupled (FIG. 1.4) at the C-terminal end, either to the 5′ end of a nucleic acid (LNA, methylphosphonate nucleic acid, thioate nucleic acid, etc.), or to the N-terminal end of a PNA.

The other VD domain is covalently coupled at its C-terminal end either to the 3′ end of a nucleic acid (LNA, methylphosphonate nucleic acid, thioate nucleic acid, etc.), or to the C-terminal end of a PNA.

The two complementary nucleotide sequences which are oriented in an anti-parallel manner pair up, creating an alpha helix that allows two protein portions to acquire a functional form. In other words, the PNA and/or nucleic acid sequences grafted onto the peptides of the VD domain act as a “zipper” type structure (“zipper” or structural PNA) and the two VD domains face each other to re-form the active site of a half-antibody (FIG. 1.5a), which is capable of targeting an intra and/or extra-cellular antigen.

This self-assembling may be used to associate, in novel assemblies, novel VD domains or VD domains obtained from existing unassociated VD domains, in order to research novel affinities. This invention also describes a method for synthesizing HAB chimeras and, finally, particular examples of use of specific HABs, in particular in combatting HIV-1 and HIV-2.

Peptide Spacers/Linkers: Polymer with 1 to 20 Carbon Atoms:

In a particular embodiment, the VD domains used conserve at least one cysteine, allowing disulfide bridges to be established between VD domains. The nucleic acids and/or the PNAs are then grafted after said cysteines, so that on hybridizing, the nucleic acids and/or grafted PNAs arrange the cysteines judiciously so that disulfide bridges are formed between the two VD chains which will form the active site of the half-antibody. Pairing of zipper type nucleic sequences means that a stable molecule is formed with a disulfide bridge having a modified redox potential. This disulfide bridge is maintained in an intracellular medium, which increases the possibility of having a functional half-antibody.

In certain embodiments, linker spacer molecules such as lysine, ethyl diamine, beta-alanine, etc., or any other hydrocarbon chain may be interposed between the nucleic acid and/or the structural PNA of the VD domain, so that once the nucleotides have been hybridized, the offset introduced by the linker spacers between the two VD domains can be used to optimize the formation of the active site of the half-antibody.

Grafting:

In a preferred embodiment, a PNA (a) is grafted at the C-terminal end of a VD domain, via the NH₂ of the side chain of a lysine bonded to the C-terminal end of said PNA (a) via a peptide linkage. The PNA (a) is thus in the C to N orientation, opposite to the N to C orientation of VD, and the lysine acts as a linker spacer. The second VD domain is linked via its C-terminal end to the N-terminal end of another PNA (b), by means of a peptide or amide linkage with one of the NH₂ groups of a lysine, said lysine being bonded, via its COOH group, to the N-terminal end of the PNA (b) complementary to the PNA (a). Thus, the PNA (b) is oriented from N to C, as is the VD.

In a particular embodiment, grafting of a PNA modified at the N-terminal or C-terminal end by a lysine or a nucleic acid modified at the 5′ or 3′ end by an NH₂ to the C-terminal end of the VD domains is carried out by the action of 2-mercapto-ethanesulfonic acid or by the action of 3-mercaptopropionic acid on the cysteine of the VDs involved in the formation of the disulfide bridge binding the heavy chain and the light chain of an antibody.

VD Peptide Domains Used:

The VD domains used to produce the chimeras may originate either from digestion of antibodies by pepsin, papain, trypsin (or any other enzyme) or from the expression of genes coding for these peptides in cellular systems such as bacteria, yeasts, insect cells or in vitro synthesis (kits, etc.), then by purification. The size of these domains may, for example, be from 10 to 300, 20 to 200 or 40 to 200 amino acid residues.

Structural “Zipper” Nucleic Acids (or Nucleotide Domains (II); FIG. 1.4; 1.5b):

The nucleic acids used to structure the half-antibody are complementary sequences with a size that may, for example, be from 3 to 100 or 5 to 60 nucleotides. These sequences may be simple nucleotide sequences, PNAs, LNAs, etc.

Cloning:

In the case of cloning VD domains, the VD domain may be fused at its C end with a peptide sequence LPXTAAAA (leucine-proline-X-threonine-alanine(4) (X=any amino acid); SEQ ID No. 2). The nucleic acid or the PNA to be grafted is functionalized at one of its ends by a poly-G, possibly via a lysine, the N-terminal end of the poly-G remaining free. The VD domain and the nucleic acid or PNA are thus fused under the action of sortase A.

The sequence LPXTAAAA may advantageously be replaced by the sequence LPXTG (leucine-proline-X-threonine-glycine, where X is any amino acid; SEQ ID NO. 7).

In a particular embodiment, it is possible to combine the VDs of different antibodies by complexing them using complementary structural PNAs in order to obtain novel antibodies directed against novel antigens. It is thus possible to screen antigens to research novel antibodies.

In a particular embodiment, the ADN/AB half-antibodies described have both the capacity to specifically reach the target cell (recognition property provided by the antibody portion) and that of delivering a biologically active domain into the interior of the cell (property provided by the added hydrophobic sequence). Thus, the chimeric molecule ensures addressing of the nucleic acid to the cell, but also its penetration into the interior of the cell.

The invention also concerns a HAB that may be used to target specific cells in order to introduce therein, for example, a biologically active nucleic domain (e.g.: PNA) directed against a particular target. This biologically active domain is grafted via a covalent bond following one of the zipper nucleic sequences described above. In this case, the active site or the HAB recognition antibody is directed against the surface antigens. This means on the one hand that the target cells can be specifically recognized, and on the other hand that penetration of a biologically active domain by bringing it to the membrane is encouraged.

In other words, the chimeric molecule is constituted by variable portions of a VD1/VD1b half-antibody (FIG. 1.5a). A structural nucleic acid (zipper) is grafted onto each VD peptide so that this nucleic acid grafted onto a peptide VD1a is complementary and anti-parallel to the structural nucleic acid (zipper) grafted onto the second peptide VD1b. The two VD peptides then form a half-antibody with its active site or recognition site. One of the two structural nucleic acids continues via a nucleotide sequence (tag) (FIGS. 1.6 to 1.8) comprising a biologically active site.

In a more particular embodiment, the half-antibody can be used to target organelles or intracellular molecules

Biologically Active Domain (or Tag Domain; FIGS. 1.6 to 1.9)

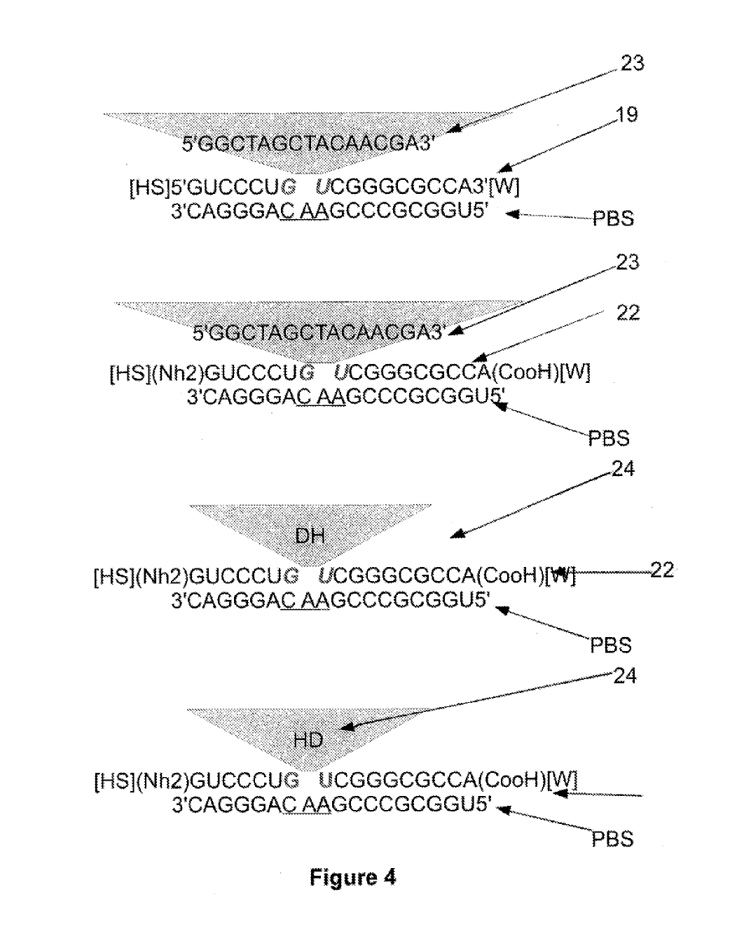

The biologically active domain may be a nucleic acid, a PNA, a mixed PNA/nucleic acid sequence, PNA/peptide and more generally a nucleotide sequence or an analog (PNA, LNA, methylphosphonates, methylsulfonates, modified nucleic acids or deoxynucleic acids or a mixture of these various molecules, etc.) a DNAzyme, a PNAzyme, or a miRNA, optionally antisense, which on entering the target cell will prevent the expression of a target sequence or will degrade it or exhibit DNAase or RNAase activity, or a combination of these two properties (FIG. 4.19).

In a particular embodiment, the biologically active domain is an antisense sequence comprising all or a portion of the tRNA^{Lys3}, for example GTCCC or GUCCCii, complementary to a portion of HIV-1 viral RNA.

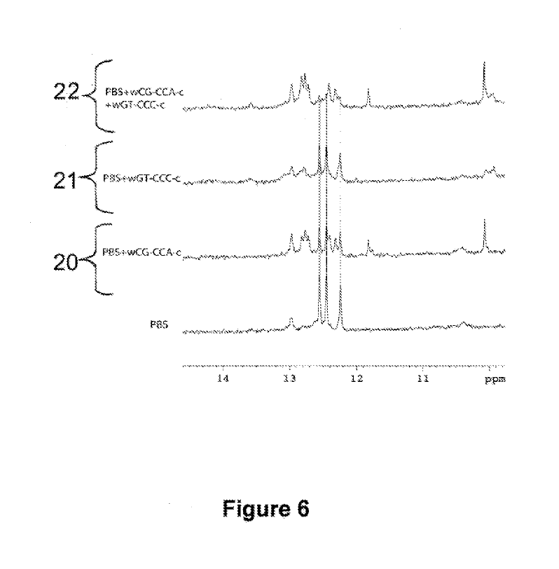

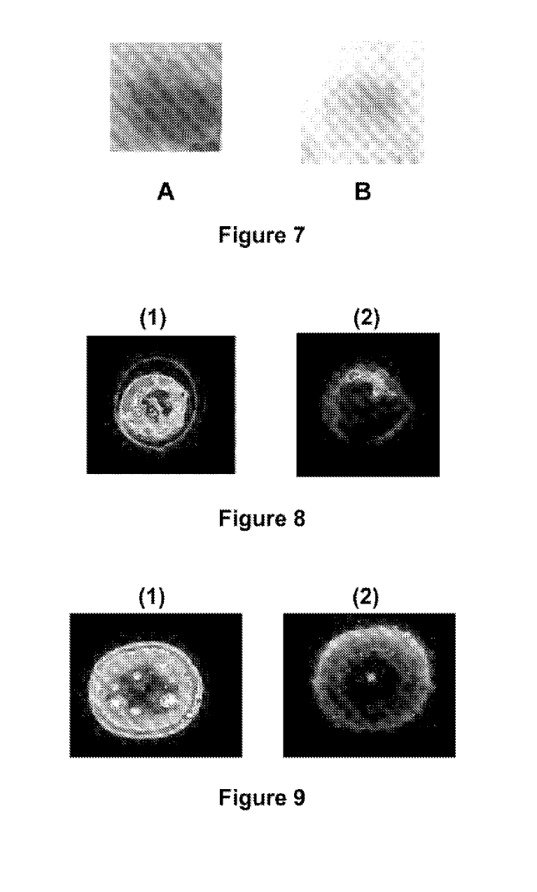

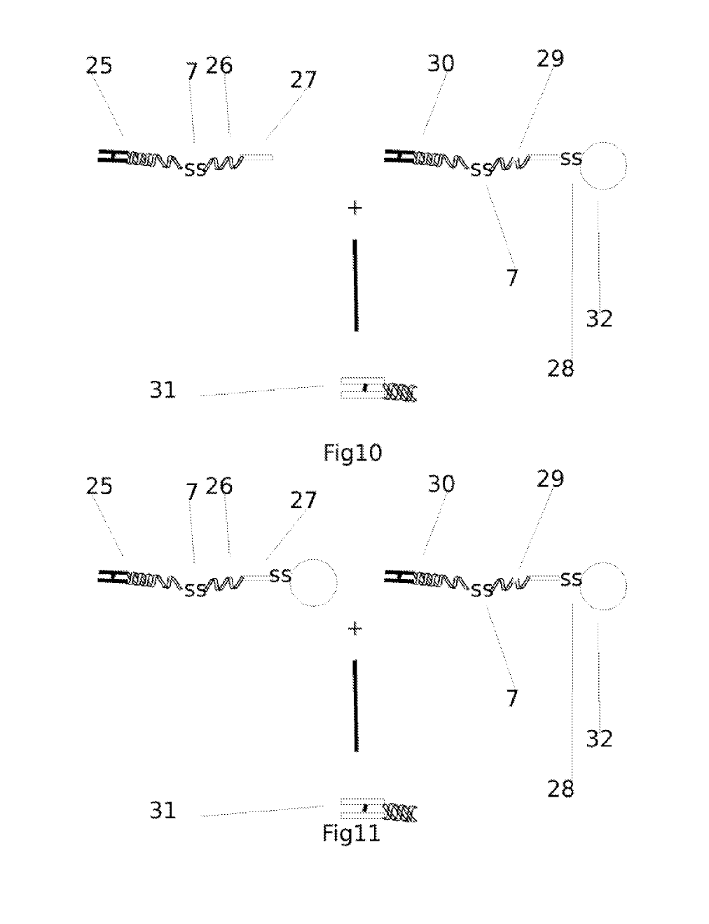

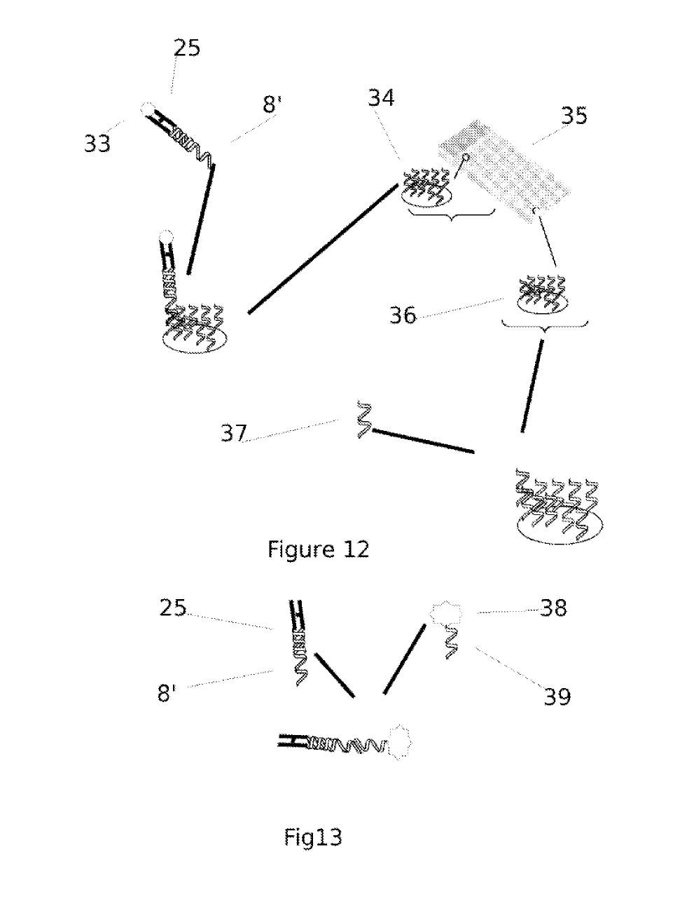

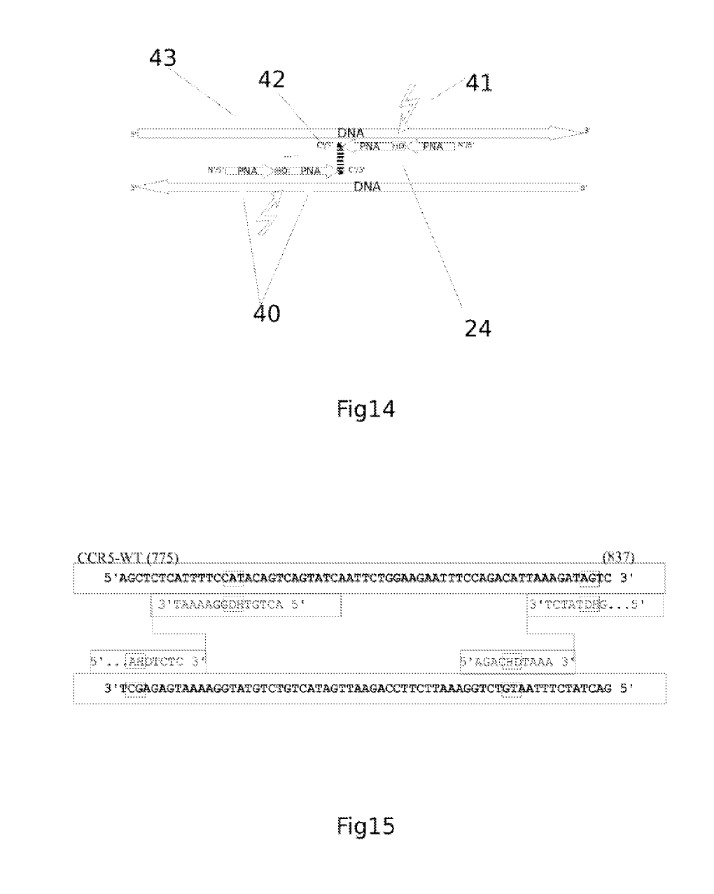

FIG. 6.21, shows the interaction, demonstrated by NMR, between a PNA w-GTCCC-c (w: tryptophan and c: cysteine, with N-terminus at 5′ and C-terminus at 3′) and the Primer Binding Site (PBS) sequences of HIV-1. The sequence forms an extremely stable complex with the PBS of HIV-1, capable of displacing the complex tRNA^{ARNtLys3}/PBS of HIV-1.

In another embodiment, the biologically active domain is an antisense sequence comprising all or a portion of the terminal sequence of tRNA_lys3, for example CGCCA, complementary to a portion of HIV-1 viral RNA.

FIG. 6.20, shows the interaction, demonstrated by NMR, between a PNA w-CGCCA-c (w: tryptophan and c: cysteine, with N-terminus at 5′ and C-terminus at 3′) and the PBS sequences of HIV-1. The sequence forms an extremely stable complex with PBS of HIV-1, capable of displacing the complex tRNAtRNALys3/PBS of HIV-1.

In a still more particular embodiment, the biologically active domain proper is a combination of two sequences, GTCCC or GUCCC, and CGCCA (FIG. 6.22) (N′ ATGGTAGAG-cysteine C′ and N′ cysteine-CTCTACCAT C′).

In a particular embodiment, the antisense sequence proper is a sequence complementary to all or a portion of the PBS region or the TAR region of HIV-1 or HIV-2 viral RNA.

In a particular embodiment, the antisense biologically active domain comprises PNAs (FIG. 6.22).

In a particular embodiment, the biologically active domain is a sequence with enzymatic activity of the DNAzyme type and complementary recognition sequences of all or a portion of the PBS sequence.

In a still more particular embodiment, the biologically active domain is constituted by DNA/PNA-zyme dimer. The PNA portion (flanking either side of the DNAzyme loop) is complementary to a target sequence of interest that may be all or a portion of the PBS sequence or TAR sequence of HIV-1 or HIV-2 (FIG. 4.22) and the DNAzyme portion, formed by 2 to 23 residues, has a loop structure (FIG. 4.23) and will bring about degradation of the target sequence because of its RNAse or DNAse activity.

In a particular embodiment, all of the enzymatic sequence is an PNA, thereby forming a PNAzyme.

In a particular embodiment, the enzymatic loop of the PNAzyme is completely or partially substituted by a motif comprising at least one amino acid such as DH (aspartic acid-histidine), HD (histidine-aspartic acid), EH (glutamic acid-histidine) or HE (histidine-glutamic acid) (FIG. 4.24).

In a particular embodiment, the PNAzyme sequence, antisense, PNA/DNAzyme proper is used alone or in combination with any portion of the HAB tag (HAB, spacer sequence, lipophilic or aromatic end).

The biologically active sequence system may be adapted to any types of virus or bacteria, in particular by targeting antibiotic resistance genes in order to inhibit the resistance and render the bacteria sensitive to the corresponding antibiotic.

In a particular embodiment, the antisense sequence proper comprises all or a portion of the RNA or DNA sequence of bacterial genes for antibiotic resistance or an antisense sequence complementary to all or a portion of said sequence.

Nucleic Spacers/Linkers (FIG. 1.6):

The grafted sequence may comprise a spacer sequence of 5 to 50 nucleotides intended to optimize the distance between the nucleic domain ii) and the biologically active domain that has to penetrate into the cell.

In a preferred embodiment, the tag sequence comprises a deoxyribonucleic acid portion acting as the spacer (FIG. 1.6). The spacer is bonded to a PNA sequence (or to another nucleic acid, which may or may not be modified) constituting the biologically active domain proper (FIG. 1.8).

The biologically active domain is, for example, bonded via one of its ends to the spacer sequence via a sequence that is labile in a reducing medium, for example a disulfide SS bridge (FIG. 1.7).

In a particular embodiment, the spacer sequence is absent and the biologically active domain is bonded to the half-antibody via a covalent bond.

In a particular embodiment, a thiol function is present at the other free end of the spacer sequence. This function can be used to graft another molecule onto the spacer sequence, for example by establishing a SS bond. This SS bond is labile in a reducing medium such as the medium in the cell cytoplasm. After reduction of the SS bridge, this bond can be used to separate the HAB and the biologically active domain. In a particular embodiment of the invention, the thiol function is introduced at the end of the spacer sequence by fusion, with said sequence, of a sequence comprising a thiol function at one of its ends. The fusion of the spacer sequence and the sequence comprising a thiol function at its free end may be carried out using a ligase.

As an example, the biologically active domain may comprise a thiol function at one of its ends in order to establish a disulfide bridge between the biologically active domain and the spacer sequence. This thiol function may be replaced by a maleimide acid so that the bond between the spacer sequence and the biologically active domain persists in a reducing medium.

Transmembrane Passage:

The target of this HAB may be extra- or intra-cellular, which implies, in this latter case, that the nucleic acid passes through the plasma membrane. This passage is rendered possible by adding a hydrophobic sequence to the free end of the nucleic acid (biologically active domain) to be transferred. This lipophilic group (FIG. 1.9) allows the biologically active domain to pass through the cell membrane more easily. In one embodiment that is even more preferred, the lipophilic group is aromatic, and can become interposed in a mixed PNA/nucleic acid helix in order to stabilize this structure formed between the antisense PNA and the nucleic acid target.

The recognition site for the HAB_tag structure binds specifically to cells comprising the target antigens; the lipophilic function grafted to the biologically active domain (antisense PNA, DNAzyme, etc.) allows the nucleotide arm, more particularly the biologically active domain to pass through the membrane. The intracellular medium is a reducing medium, and so the SS bridges linking the biologically active domain to the spacer sequence are reduced, thereby releasing the biologically active domain into the interior of the target cell.

As an example, the antisense PNA then complexes with sense target sequences (RNA in the cytoplasm or DNA in the nucleus) and blocks the possibilities of expression of the target sequence (for transcription, retrotranscription, replication, and/or translation). The aromatic molecule, such as tryptophan, introduced at the end of the antisense PNA stabilizes the PNA/DNA or RNA duplex that forms and thus improves the blocking efficiency by the PNA.

Antibody or VD Peptide Domains

In a particular embodiment, the antibody used to produce the HAB_tag is an antibody recognizing a protein of a viral or bacterial envelope.

In a particular embodiment, the antibody used to produce the HAB_tag is capable of binding to the viral particle.

In a particular embodiment, the antibody used to produce the HAB_tag is the antibody anti gp120-17b. The HAB_tag-gp120-17b can bind to viral particles and is led by them to the target cells of the virus by means of CD4/gp120 interactions. After binding virions onto the targets, the nucleic arm of the HAB-tag can pass through the cell membrane due to the lipophilic function grafted to the end of the biologically active domain. After passing through the cell membrane, the SS bridge binding the spacer sequence and the biologically active domain is reduced in the cytosol, releasing the biologically active domain into the cell. The biologically active sequence blocks or blocks and degrades the viral sequence, depending on whether the biologically active domain is antisense or antisense/PNAzyme.

In accordance with a particular embodiment, the antibody used for synthesis of the half-antibody is constituted by an anti-retrotranscriptase antibody, in particular anti-retrotranscriptase HIV-1 or HIV-2. In this configuration, the half-antibody is bonded to the biologically active domain via a bond that is persistent in a reducing medium, for example via a maleimide acid. A spacer sequence may optionally be interposed between the half-antibody and the biologically active domain, the bonds connecting HAB, spacer sequence and biologically active domain being persistent in a reducing medium. The biologically active domain is preferably a PNAzyme, DNAzyme or antisense PNA sequence. The HAB_tag constituted thereby forms a complex with the reverse transcriptase, while the biologically active domain degrades and/or blocks viral RNA.

The HAB_tag may be included (in particular encapsidated) in microspheres or liposomes; more particularly, the HAB tags will be included in liposomes constituted by viral membrane.

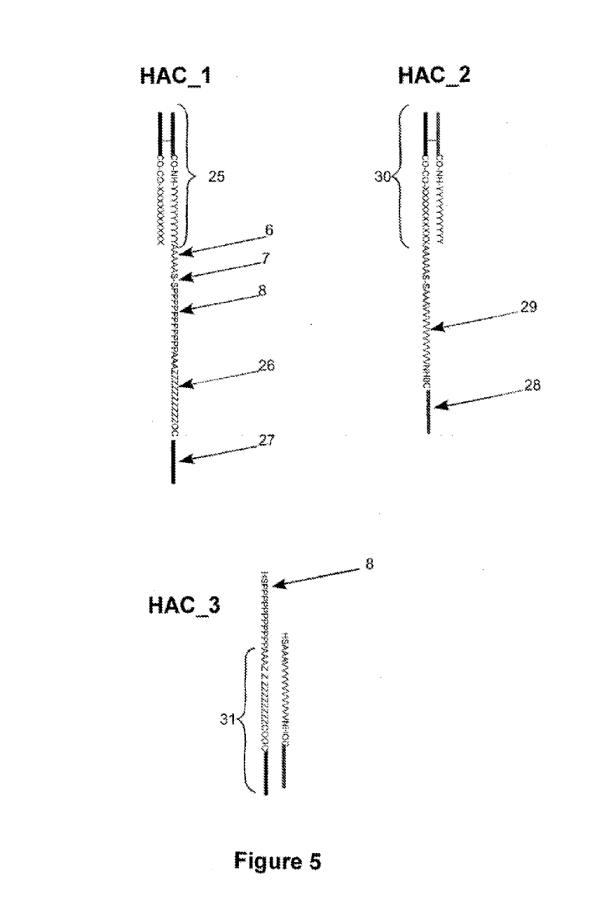

The invention also concerns a system comprising (see FIG. 5):
1) a first HAB (HAB_{—}1; FIG. 5.25), directed, for example, against an antigen present on the surface of cells or a viral antigen and comprising or constituted by:
   two VD domains (VD1a and VD1b), respectively bonded to:
   two complementary, antiparallel zipper sequences (S1 and S2),
   a spacer bonded to a zipper sequence of HAB_{—}1, bonded via one of its ends and via a SS bridge to:
   a biologically active domain (FIG. 5.8), bonded via a covalent bond to:
   a structural nucleic acid, S3 (FIG. 5.26), of a third HAB (HAB_{—}3),
   the other extremity of the structural sequence (S3) being bonded to:
   a VD domain (VD2a; FIG. 5.27);
2) a second HAB (HAB_{—}2) directed, for example, against an antigen also present on the surface of cells and comprising or constituted by:
   two VD domains (VD3a and VD3b), respectively bonded to:
   two complementary, antiparallel zipper sequences (S5 and S6),
   a spacer comprising a disulfide bridge or bonded via a disulfide bridge to:
   a complementary structural nucleic acid, S4 (FIG. 5.29), antiparallel to S3, bonded to:
   a VD domain (VD2b; FIG. 5.28).
3) in a cell comprising HAB_{—}1 and HAB_{—}2, a third HAB, HAB_{—}3 is formed. This comprises:
   two VDs (VD2a and VD2b), bonded to:
   two complementary, antiparallel structural nucleic acids, S3 and S4, (respectively derived from HAB_{—}1 and HAB_{—}2),
   S3 being bonded via a covalent bond to:
   A biologically active domain derived from HAB_{—}1 (FIG. 5.8).

In accordance with a particular embodiment, HAB_{—}1 is directed against the protein gp120, HAB_{—}2 is directed against the protein CD4.

In accordance with a particular embodiment, VD2a and VD2b form an anti-retrotranscriptase recognition site HAB_{—}3 (FIG. 5.31) when the two structural nucleic acids S3 and S4 hybridize.

This system may be put together in various manners to deliver, inside a cell, the two portions of a half-antibody, which may then self-assemble in the cell. It is also possible to use viral particles to specifically deliver the half-antibodies and their biologically active domain to the cell.

Preparation of Chimeric Molecules by Fusing Disulfide Bridges.

a) Reactor

The antibody underwent an electrochemical reduction carried out in a cooled reactor. The reactor was composed of a vertical tube of brass 2 mm in diameter and 1.5 cm in length containing the antibody solution to be electro-reduced. The lower end of the tube was connected to the positive terminal of a generator via a mixed electrode composed of a needle filled with and surrounded by a 20% duracryl gel (124 μL of 30% duracryl (Sigma)+30 μL of Tris 1.5M, pH 8.9+43.6 μL H₂O+50 μL of electro-reduction buffer+5 μL of 10% APS+2 μL of Temed) contained in a 1.5 cm Tycon tube (Norton), sealed on one side using a Bunsen burner flame. The gel electrode allowed electrolytes to pass, but limited the diffusion of antibodies or their reduced forms. The upper end of the reactor was connected to the negative pole of the generator via a needle so as to prevent any contact between the walls of the reactor and the electrode by means of a pipette tip which guided the needle into the interior of the reactor. The reactor was surrounded by a cooling system constituted by another tube of brass twisted to form a coil in which water circulated at the temperature of melting ice, under the action of a peristaltic pump. The parameters used for reduction of the antibody to half-antibody were: +100V, 2 mA, 10 sec.

b) Functionalization of Half-Antibodies by Oxidation of Thiol Functions.

A solution of 4 μL of anti-BSA antibody, 0.5 μg/μL, 4 μL of 0.12% β-mercaptoethanol buffer and 6 μL of the Tag_C sequence, 500 ng/μL (^{5′}HS-TCAAGTAGCTTCAGAATCTTTTCCCCCC^{3′} SEQ ID No. 3), was used to functionalize the thiol functions (SH) released on the antibodies after reduction by electro-oxidation at −100V, 2 mA, for 10 seconds.

Detection by Fluorescence

The tag_C sequences of the chimeras were tagged using a complementary nucleotide sequence (tag_D=^{5′}CTGAAGCT³_cysteine) of the tag_C sequence and functionalized at 3′ by a cyanine 3 fluorophore. 15 μL of the tag_D sequence (0.3 μg/μL) was added to 10 μL of a solution of chimeras functionalized by a tag_C sequence and diluted (1/1000) in milk/TBS1X. In this solution, a deposit of an antigen on nitrocellulose membrane was incubated overnight at 4° C. After washing three times with TBS 1X for 5 minutes to eliminate aspecific complexing, the membrane was denatured with 50 μL of 2.5% β-mercaptoethanol at a temperature of 95° C. for 20 minutes. 2 μL of supernatant was deposited on a glass slide. The deposit was dried for 20 minutes in the open air and away from the light, then the slide was scanned (Packard Bioscience) at 455 nm.

A negative control was carried out at the same time: a membrane comprising a deposit of antigen was incubated with 15 μL of tag_D and 1 mL of milk/TBS 1X at 4° C. overnight with agitation. The membrane was subjected to the same protocol as before. 2 μL from denaturing it was also deposited on a glass slide and scanned to determine the aspecific complexing.

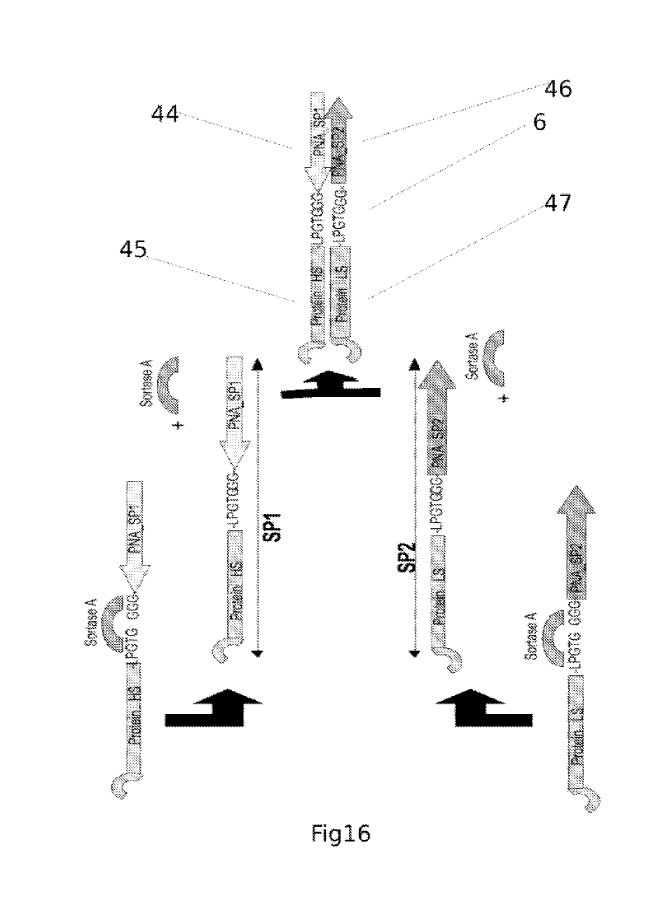

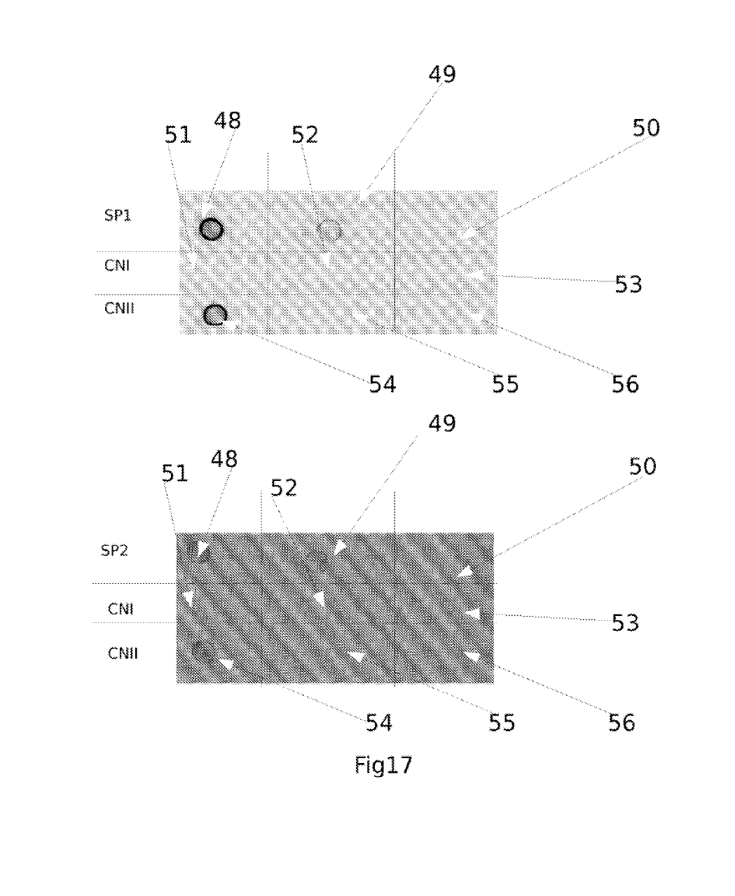

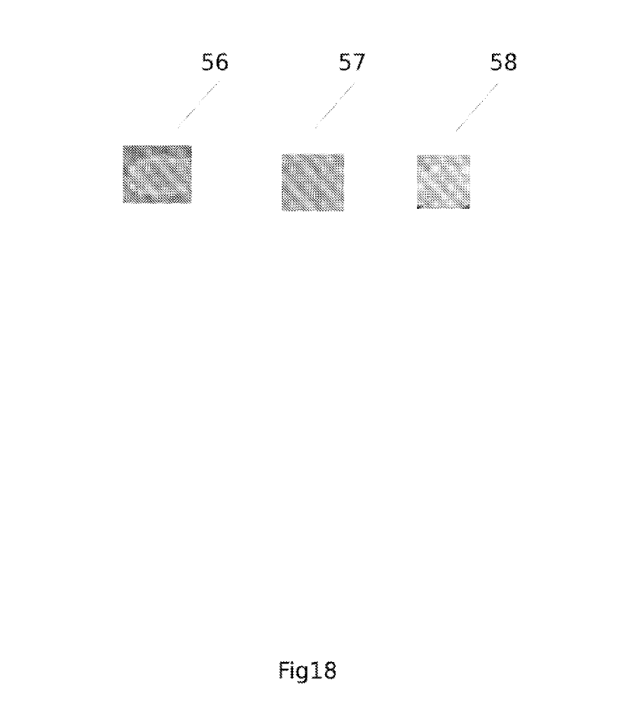

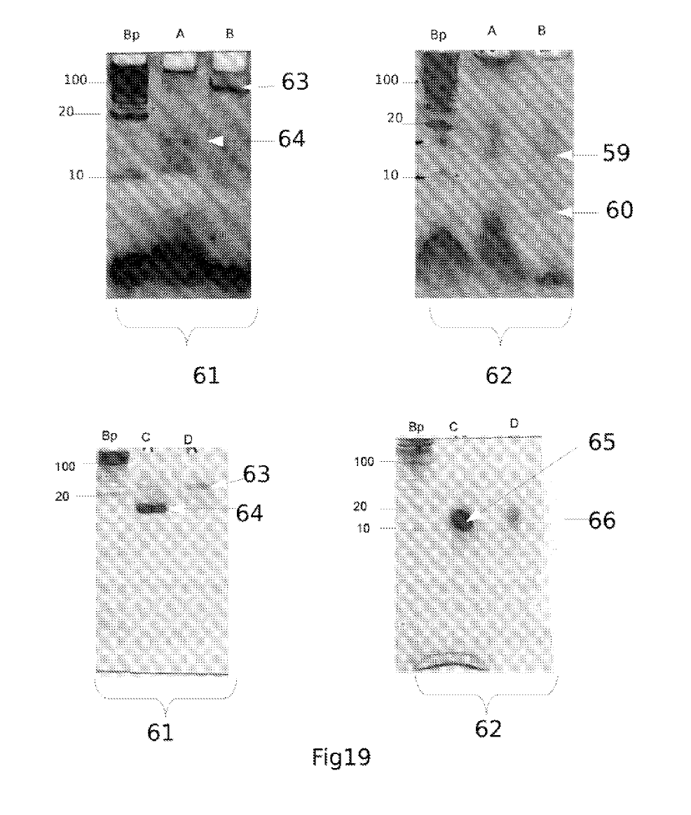

C) Fusion of (i) VD and (ii) Zipper Domains Using an Enzymatic Transpeptidation Protocol Using Sortase a (FIG. 16)

### c.1) Sortase A

The plasmid pLLC092 (http://isisbiolab.dabbledb.com/publish/laboratorystocks/ff0e3c28-ca9a-4280-95ce-f170e7f7a39a/allentries.txt) containing the sequence for sortase contiguous with a histidine tag was transfected into BL21(DE3) competent cells (Biolabs) under the control of a Lac promoter induced by IPTG (isopropyl β-D-1-thiogalactopyranoside). The expression of sortase A was then induced by IPTG (1 mM). The enzyme sortase A was then purified using the Ni-NTA Fast Start Kit (Qiagen).

### c.2) HS-LPGTG & LS-LPGTG Proteins

(I-A) HS-LPGTG:
(SEQ ID NO. 8)
5′CAGGTGCAGCTGAAAGAATCTGGTCCGGGTCTGGTGGCGCCGTCTCA
GTCTCTGTCTATTACCTGCACCGTGTCTGGTTTTTCTCTGACCGGTTAT
GGTGTGAACTGGGTGCGTCAGCCGCCGGGTAAAGGTCTGGAATGGCTGG
GTATGATTTGGGGTGATGGTAACACCGATTATAACTCTGCGCTGAAATC
TCGTCTGTCTATTTCTAAAGATAACTCTAAATCTCAGGTGTTTCTGAAA
ATGAACTCTCTGCATACCGATGATACCGCGCGTTATTATTGCGCGCGTG
AACGTGATTATCGTCTGGATTATTGGGGTCAGGGTACCACCCTGACCGT
GTCTTCTGCGTCTACCACCCCGCCGTCTGTGTTTCCGCTGGCGCCGGGT
TCTGCGGCGCAGACCAACTCTATGGTGACCCTGGGTTGCCTGGTGAAAG
GTTATTTTCCGGAACCGGTGACCGTGACCTGGAACTCTGGTTCTCTGTC
TTCTGGTGTGCATACCTTTCCGGCGGTGCTGCAGTCTGATCTGTATACC
CTGTCTTCTTCTGTGACCGTGCCGTCTTCTCCGCGTCCGTCTGAAACCG
TGACCTGCAACGTGGCGCATCCGGCGTCTTCTACCAAAGTGGATAAAAA
AATTGTGCCGCGTGATTGCCTGCCGGGTACCGGT 3′ → 669 bases.
(I-B) LS-LPGTG:
(SEQ ID NO. 9)
5′GATATTCAGATGACCCAGTCTCCGGCGTCTCTGTCTGCGTCTGTGGG
TGAAACCGTGACCATTACCTGCCGTGCGTCTGGTAACATTCATAACTAT
CTGGCGTGGTATCAGCAGAAACAGGGTAAATCTCCGCAGCTGCTGGTGT
ATTATACCACCACCCTGGCGGATGGTGTGCCGTCTCGTTTTTCTGGTTC
TGGTTCTGGTACCCAGTATTCTCTGAAAATTAACTCTCTGCAGCCGGAA
GATTTTGGTTCTTATTATTGCCAGCATTTTTGGTCTACCCCGCGTACCT
TTGGTGGTGGTACCAAACTGGAAATTAAACGTGCGGATGCGGCGCCGAC
CGTGTCTATTTTTCCGCCGTCTTCTGAACAGCTGACCTCTGGTGGTGCG
TCTGTGGTGTGCTTTCTGAACAACTTTTATCCGAAAGATATTAACGTGA
AATGGAAAATTGATGGTTCTGAACGTCAGAACGGTGTGCTGAACTCTTG
GACCGATCAGGATTCTAAAGATTCTACCTATTCTATGTCTTCTACCCTG
ACCCTGACCAAAGATGAATATGAACGTCATAACTCTTATACCTGCGAAG
CGACCCATAAAACCTCTACCTCTCCGATTGTGAAATCTTTTAACCGTAA
CGAATGCCTGCCGGGTACCGGT 3′ → 657 bases.

The above two sequences (I-A) HS-LPGTG and (I-B) LS-LPGTG, corresponding respectively to the variable portion of the heavy chain and of the light chain of the anti-HEL (Hen Egg Lysozyme) antibody, were cloned into the vector pEXPS-NT (Invitrogen) by Proteigenix. The vectors were transfected separately into competent Top10 one shot E. coli bacteria (Invitrogen). Purification of the plasmids was carried out with the QIAfilter Plasmid Midi Kit (Qiagen). Expression of the two proteins HS-LPGTG & LS-LPGTG was carried out using the Expressway Cell-free E. coli expression system (Invitrogen). The two synthesized proteins were then purified using the Ni-NTA Fast Start Kit (Qiagen).

Two PNA zippers (domain ii-A SP1 & (ii-B) SP2) with respective sequences ATGGTAGAG and CTCTACCAT extended by the linkers N′GlyGlyGly(CO)NH(CH2)4(NH2)CH(COO) and NH₂—CH(CONH₂—(CH₂)₄NH(CO)GlyGlyGly N′), were synthesized in order to obtain

SP1
(N′GlyGlyGly(CO)NH(CH₂)₄(NH₂)CH(CO)
NHATGGTAGAGTTTTTC′)
and
SP2
(N′ CTCTACCAT-CONH-CH(CONH₂)-(CH₂)₄NH(CO)
GlyGlyGly N′).

A sequence TTTTTC was added to the end of SP1 in order to be able to detect the chimera formed, tagging it with a fluorescent nucleic acid marker GAAAAA.

SP1 and SP2 were taken up in a 10% N-methyl-2-pyrrolidone (NMP) solution to a final concentration of 100 μM.

Test-I: A solution of 50 uL of SP1 and a solution of 50 uL of SP2 were prepared at a final concentration of 20 uM containing 20 uM of HS-LPGTG (I-A), and respectively 20 uM of LS-LPGTG (I-B), the enzyme SrtA, 20 uM, Tris-HCl, pH 7.5, 50 mM, NaCl, 150 mM and CaCl₂, 5 mM.
Four negative controls were also produced (FIG. 17):
CNI-sp1 (solution without PNA), 50 uL of solution containing HS-LPGTG (I-A), 20 uM, and the enzyme SrtA, 20 uM.
CNII-sp1 (solution without srtA), 50 uL of solution containing HS-LPGTG (I-A), 20 uM and the PNA-SP1, 20 uM.
CNI-Sp2 (solution without PNA), 50 uL of solution containing LS-LPGTG (I-B), 20 uM, and the enzyme SrtA, 20 uM.
CNII-SP2 (solution without srtA), 50 uL of solution containing LS-LPGTG (I-B), 20 uM SP2, 20 uM.

The tubes were incubated for 24 h at 37° C.

### c.3 Purification Using Magnetic Beads and Actev Enzyme

25 uL of Ni-NTA magnetic agarose beads (Qiagen) were precipitated, the supernatant was removed. 50 uL of the solution to be purified+100 uL of protein binding buffer (50 mM NaH₂PO₄, 300 mM NaCl, 20 mM imidazole, pH8) were added to these beads. It was incubated for 1 h30 on a wheel at ambient temperature and at 7 rpm. The beads recovered from supernatant No 1 were precipitated. Two washes of beads with 150 uL of protein binding buffer. Cleavage of histidine tag with the enzyme AcTev protease (Invitrogen), 10 uL of 5×Tev buffer, 5 uL of 0.01M DTT, 0.5 uL of AcTev protease enzyme, 34.5 uL of H₂O, incubation for 4 h at 30° C. Precipitation of beads, recovery of supernatant n°2 (containing the molecules that had undergone transpeptidation). Beads washed twice with 150 uL of protein binding buffer. Elution of proteins still present on beads with 50 uL of elution protein buffer (50 mM NaH₂PO₄, 300 mM NaCl, 250 mM imidazole, pH8) for 1 h at ambient temperature on a wheel and at 7 rpm. Precipitation of beads, recovery of supernatant n°3.

### c.4 Detection of Fusion (see FIG. 17).

For SP1 and SP2 respectively, 2 uL of each supernatant (test, CNI, CNII) recovered was deposited on a Protran nitrocellulose membrane (Whatman). The deposits were dried for 20 minutes at ambient temperature. Saturation with a solution of 5% milk/TBS1X, pH7.6 at 4° C. for 20 minutes. Hybridization of membrane overnight at 4° C. with light agitation in a 5% milk/TBS1X pH7.6 buffer containing 50 μM of sequences, fluorescently tagged with Cy-3 and 6-FAM respectively, complementary to PNA-SP 1 or to PNA-SP2 respectively. Three washes of 15 minutes of the membrane with a solution of 5% milk/TBS1X, pH7.6. Scan using Typhoon 8600 (Amersham Biosciences).

The results shown demonstrated fusion between the molecule SP1 and SP2 with the peptides HS-LPGTG and LS-LPGTG respectively to obtain SP1(I-II) and SP2(I-II).

d) Self-Assembly of Chimeras

d.1) Hybridization of SP1 (i-ii) Molecules with SP2 (I-II) molecules 12.5 uL of supernatant N°2 produced in C.3 (see above) of SP1(I-II) molecules and 12.5 uL of supernatant N°2 produced in C.3 (see above) of SP2 (I-II) molecules+25 uL of neutral buffer (200 uL of 99% glycerol, 33 uL of Tris 3M, pH6.8, 267 uL of H₂O) were hybridized for 1 h at 37° C.

### d.2) Verification of Functionality of the Molecule (See FIG. 18).

5 times 2 μL of the solution obtained at D.1 were deposited onto a Protran nitrocellulose membrane (Whatman), drying for 10 min between each deposit to form a deposition zone or spot.

Two controls were produced:

a) 2ul of anti-HEL antibody (Tebu-bio), 0.1 ug/uL, and b) 5 times 2 μL of anti-HEL antibody (Tebu-bio), 10 uM (concentration comparable to the paired molecule) were respectively deposited on the same membrane, drying for 10 min between each deposit at ambient temperature in order to respectively form two other deposition zones.

The nitrocellulose membrane was then saturated with a solution of superblocking buffer (Thermofisher) at 4° C. for 20 minutes, then hybridized overnight at 4° C. with light agitation, with a solution containing 250 nM of HEL antigen (Fluka) labeled with Dylight488 (Thermofisher) in superblocking buffer. Three 15 minute washes of the membrane with superblocking buffer. Scan using Typhoon 8600 (Amersham Biosciences).

The results (FIG. 18) demonstrate that the chimeric protein captures its antigen and is thus functional after the step for self-assembly by hybridization.

e) PNAzyme Example: Cleaving of PBS (HIV-1) (see FIG. 19)

A solution of “Primer Binding Sequence” RNA (or PBS) from HIV-1 with 18 bases (5′ UGGCGCCCGAACAGGGAC 3; SEQ ID NO.10), 10 uM in H₂O, was mixed mole for mole, either with a solution of PNAzyme (N′ TCCCTGHDTC C′), 10 uM in 10% NMP, or with a 10% NMP solution. The mixtures were incubated for 4 h at 37° C. The mixtures were deposited either onto a native gel containing 20% acrylamide (1.5 mL of TBE10X, 7.5 mL of acrylamide/bis, 19:1 (Biorad), 6 mL of H₂O, 87.5 uL of 10% APS and 7.5 uL of Temed (Sigma)); or in a denaturing gel containing 20% acrylamide (7.2 g urea (Sigma), 1.5 mL de TBE10X, 7.5 of acrylamide/bis, 19:1 (Biorad), 6 mL of H₂O, 87.5 uL of 10% APS and 7.5 uL of Temed (Sigma)). After migration at 100V and 200 mA for 1 h30, the gels were revealed with ethidium bromide.

Analysis of the gels showed that the PNAzyme hybridizes with PBS and is capable of cleaving the PBS molecule.

### REFERENCES

David Baulcombe. 2002. An RNA Microcosm. Science, 297: 2002-2003.
Rong Fan, Ophir Vermesh, Alok Srivastava, Brian K H Yen, Lidong Qin, Habib Ahmad, Gabriel A Kwong, Chao-Chao Liu, Juliane Gould, Leroy Hood & James R Heath. 2008. Integrated barcode chips for rapid, multiplexed analysis of proteins in microliter quantities of blood. Nature Biotechnology. Vol. 26, No. 12: 1373-1378.
Muthiah Manoharan, Kathleen L. Tivel and P. Dan Cook. 1995. Lipidic nucleic acids. Tetrahedron Letters. Vol. 36, No. 21: 3651-3654.
Indriati Pfeiffer and Fredrik Höök. 2004. Bivalent Cholesterol-Based Coupling of Oligonucletides to Lipid Membrane Assemblies. J. Am. Chem. Soc. 126 (33): 10224-10225.
Snehlata Tripathi, Binay Chaubey, Sabyasachi Ganguly, Dylan Harris, Ralph A Casale, and Virendra N. Pandey. 2005. Anti-HIV-1 activity of anti-TAR polyamide nucleic acid conjugated with various membrane transducing peptides. Nucl. Acids Res. Vol. 33, No. 13: 4345-4356.

1. A chimeric molecule, termed a “half-antibody”, characterized in that it comprises or consists of two chimeric molecules A and B, each comprising or consisting of:
(i) a characteristic polypeptide domain of a variable domain (or VD) of a heavy chain or of a light chain of an antibody, this polypeptide domain being positioned at one end in the chimeric molecules A and B, the polypeptide domain (i) of one of the two chimeric molecules, A or B, being characteristic of a VD of a light chain of an antibody and the polypeptide domain (i) of the other chimeric molecule, respectively B or A, being characteristic of a VD of a heavy chain of an antibody; and
(ii) a single stranded nucleotide domain consisting of a polynucleotide, in particular a DNA or a RNA, or an analog of a polynucleotide, in particular a peptide nucleic acid (PNA), a locked nucleic acid (LNA), a methylphosphonate nucleic acid or a thioate nucleic acid, the nucleotide domain of A and that of B being capable of pairing into a double stranded structure, for example in a hydric medium and in particular in a reducing medium; and
(iii) if appropriate, one or more binding molecule(s) (or linker(s)), and in particular a linker binding domains (i) and (ii) of a chimeric molecule A or B, said linker(s) comprising or consisting of a hydrocarbon chain preferably comprising 1 to 20 carbon atoms, and comprising or consisting of one or more element(s) selected from a peptide or a polypeptide, a polynucleotide or an analog, a polycarbon chain, ethylenediamine, polylysine, beta-alanine, and a sugar; the polypeptide domain (i) and the nucleotide domain (ii) of each of the chimeric molecules A and B being bonded via a covalent bond, for example via a NH₂ group, in particular a NH₂ group of a linker, and the domain (ii) of the chimeric molecule A and that of the chimeric molecule B being paired into a double stranded structure in a hydric medium, in particular a reducing medium.

2. A half-antibody as claimed in claim 1, in which:
in the chimeric molecule A, the C-terminal end of the polypeptide domain (i) is covalently bonded to the 3′ end of a nucleic acid or to the C-terminal end of a PNA, for example via the NH₂ group of the side chain of a lysine bonded to the C-terminal end of said PNA via a peptide linkage; and/or
in the chimeric molecule B, the C-terminal end of the polypeptide domain (i) is covalently bonded to the 5′ end of the nucleic acid or to the N-terminal end of a PNA, for example via a peptide linkage or amide with one of the NH₂ groups of a lysine, said lysine being bonded, via its COOH group, to the N-terminal end of said PNA.

3. A half-antibody as claimed in claim 1, in which the chimeric molecule A and/or the chimeric molecule B further comprise(s) one or more element(s) selected from:
a tag domain and/or a biologically active domain, which is a polynucleotide or an analog comprising or consisting of a tag sequence and/or a biologically active sequence that can be used to block the expression of target genes, in particular in the interior of a cell, for example a polynucleotide or an analog selected from the following elements:
a nucleic acid, in particular an antisense nucleic acid or a nucleic acid with endonuclease activity, for example a ribonuclease or deoxyribonuclease activity;
a PNA, in particular an antisense PNA or a PNA with endonuclease activity (PNAzyme), for example ribo- or deoxyribonuclease activity;
a LNA, a methylphosphonate nucleic acid, a nucleic acid methylsulfonate, a modified nucleic acid or deoxynucleic acid, a DNAzyme, a RNAzyme or a PNAzyme, a micro RNA (miRNA), a mixed PNA/nucleic acid or PNA/peptide sequence, or a DNA/PNA-zyme dimer; and
a combination of at least two of these elements;
said tag domain or the biologically active domain being bonded to the free end of the nucleotide domain (ii) of the chimeric molecule A or B or to the free end of said linker bonded to the nucleotide domain (ii) of the chimeric molecule A or B, said linker being as defined, and bonding of said tag domain or of said biologically active domain preferably occurring via a bond that is labile in a reducing medium, for example a disulfide bridge, or via a bond that persists in a reducing medium, for example a bond established with a maleimide acid; and
a lipophilic element, in particular a sequence, a chemical group or a lipophilic chemical compound, which preferably comprises one or more aromatic compound(s) and/or group(s) containing one or more tryptophan residues, said lipophilic element preferably being positioned at the free end of the chimeric molecule A or B.

4. A half-antibody as claimed in claim 3, in which:
the first chimeric molecule, A or B, comprises or consists of:
said polypeptide domain (i), and the following domains, positioned in succession in the following order, starting from the C-terminal end of the polypeptide domain (i):
if appropriate, said linker;
said nucleotide domain (ii);
if appropriate, said linker, bonded, preferably via a bond that is labile in a reducing medium, for example a disulfide bridge or via a bond that persists in a reducing medium, for example a bond established with a maleimide acid, to:
said tag domain or said biologically active domain; and
if appropriate, said lipophilic element; and
the second chimeric molecule, respectively B or A, comprises or consists of:
said polypeptide domain (i), this domain and the polypeptide domain (i) of the first chimeric molecule being characteristic of VDs of the same antibody, one of these domains being characteristic of a VD of the light chain of a given antibody and the other being characteristic of a VD of the heavy chain of the same antibody;
and the following domains, positioned in succession in the following order, starting from the C-terminal end of the polypeptide domain (i):
if appropriate, said linker; and
said nucleotide domain (ii).

5. A half-antibody as claimed in claim 3, in which:
the first chimeric molecule, A or B, comprises or consists of:
said polypeptide domain (i), and the following domains, positioned in succession in the following order, starting from the C-terminal end of polypeptide domain (i):
if appropriate, said linker;
said nucleotide domain (ii), termed S1;
if appropriate, said linker, bonded, for example, via a bond that is labile in a reducing medium, in particular a disulfide bridge, to:
said tag domain or said biologically active domain, bonded, for example, via a covalent bond, to:
if appropriate, said linker;
a second nucleotide domain termed S3, which comprises or consists of a polynucleotide, in particular a DNA or a RNA, or an analog of a polynucleotide, in particular a PNA, a LNA, a methylphosphonate nucleic acid or a thioate nucleic acid, and which is capable of pairing into a double stranded structure with a complementary nucleotide domain termed S4, for example in an aqueous medium, in particular in a reducing medium; and
if appropriate, said linker;
the domain S3 or said linker being bonded to the C-terminal end of a second polypeptide domain, termed VD2a, which is characteristic of a VD of a heavy chain or of a light chain of an antibody, VD2a and the polypeptide domain (i) of the chimeric molecule A or B preferably being characteristic of VDs of distinct antibodies; and
if appropriate, said lipophilic element, said lipophilic element being bonded to VD2a via a bond that is labile in a reducing medium, for example a disulfide bridge, or via said linker, said linker comprising a bond that is labile in a reducing medium or being bonded to VD2a via a bond that is labile in a reducing medium;
the second chimeric molecule, respectively B or A, comprises or consists of:
said polypeptide domain (i), this domain and the polypeptide domain (i) of the first chimeric molecule being characteristic of VDs of the same antibody, one of these domains being characteristic of a VD of the light chain of a given antibody and the other being characteristic of a VD of the heavy chain of the same antibody;
and the following domains, positioned in succession in the following order, starting from the C-terminal end of the polypeptide domain (i):
if appropriate, said linker; and
said nucleotide domain (ii), termed S2, capable of pairing into a double stranded structure with S1 in an aqueous medium, in particular a reducing medium.

6. A half-antibody as claimed in claim 3, in which:
the first chimeric molecule, A or B, comprises or consists of:
said polypeptide domain (i), and the following domains, positioned in succession in the following order, starting from the C-terminal end of the polypeptide domain (i):
if appropriate, said linker;
the nucleotide domain (ii), termed S5;
if appropriate, said linker, said linker comprising, if appropriate, a bond that is labile in a reducing medium, for example a disulfide bridge, or being bonded via a bond that is labile in a reducing medium, for example a disulfide bridge, to:
a second nucleotide domain termed S4, which comprises or consists of a polynucleotide, in particular a DNA or a RNA, or an analog of a polynucleotide, in particular a PNA, a LNA, a methylphosphonate nucleic acid or a thioate nucleic acid, and which is capable of pairing into a double stranded structure with a complementary nucleotide domain termed S3, for example in a hydric medium, in particular in a reducing medium; and
if appropriate, said linker,
the domain S4 or said linker being bonded to the C-terminal end of a second polypeptide domain, termed VD2b, which is characteristic of a VD of a heavy chain or of a light chain of an antibody, VD2b and the polypeptide domain (i) of the first chimeric molecule being characteristic of VDs of distinct antibodies; and
if appropriate, said lipophilic element, said lipophilic element being bonded to VD2b via a bond that is labile in a reducing medium, for example a disulfide bridge, or via said linker, said linker comprising a bond that is labile in a reducing medium or being bonded to VD2a via a bond that is labile in a reducing medium; and
the second chimeric molecule, respectively B or A, comprises or consists of:
said polypeptide domain (i), this domain and the polypeptide domain (i) of the first chimeric molecule preferably being characteristic of VDs of the same antibody, one of these domains being characteristic of a VD of the light chain of a given antibody and the other being characteristic of a VD of the heavy chain of the same antibody, and the following domains, positioned in succession in the following order, starting from the C-terminal end of the polypeptide domain (i):
if appropriate, said linker; and
said nucleotide domain (ii), termed S6, which is capable of pairing into a double stranded structure with S5 in an aqueous medium, in particular a reducing medium.

7. A half-antibody as claimed in claim 1, in which the polypeptide domains (i), in particular the polypeptide domains (i), are characteristic of VD domains of one or more antibodies directed:
against an antigen present on the surface of mammalian cells, for example the CD4protein; or
against a viral antigen, in particular a protein of a viral envelope, for example the outer envelope protein of a HIV virus; or
against a bacterial antigen, in particular a bacterial envelope protein; or
against a retrotranscriptase, in particular a retrotranscriptase of HIV-1 or HIV-2.

8. A half-antibody as claimed in claim 3, in which the tag domain and/or the biologically active domain comprises or consists of two PNAzymes, in particular two PNAzymes comprising a HD (histidine-aspartic acid) or DH (aspartic acid-histidine) motif, said PNAzymes being bonded via their N-terminal or C-terminal ends via the same linker.

9. A vesicle, in particular a liposome, and more particularly a liposome constituent of a viral membrane, for example a membrane from particles of the HIV-1 or HIV-2 virus, said vesicle comprising one or more half-antibodies as defined in claim 1, said vesicle comprising, for example:
a half-antibody in which:
the first chimeric molecule, A or B, comprises or consists of:
said polypeptide domain (i), and the following domains, positioned in succession in the following order, starting from the C-terminal end of polypeptide domain (i):
if appropriate, said linker;
said nucleotide domain (ii), termed S1;
if appropriate, said linker, bonded, for example, via a bond that is labile in a reducing medium, in particular a disulfide bridge, to:
said tag domain or said biologically active domain, bonded, for example, via a covalent bond, to:
if appropriate, said linker;
a second nucleotide domain termed S3, which comprises or consists of a polynucleotide, in particular a DNA or a RNA, or an analog of a polynucleotide, in particular a PNA, a LNA, a methylphosphonate nucleic acid or a thioate nucleic acid, and which is capable of pairing into a double stranded structure with a complementary nucleotide domain termed S4, for example in an aqueous medium, in particular in a reducing medium; and
if appropriate, said linker;
the domain S3 or said linker being bonded to the C-terminal end of a second polypeptide domain, termed VD2a, which is characteristic of a VD of a heavy chain or of a light chain of an antibody, VD2a and the polypeptide domain (i) of the chimeric molecule A or B preferably being characteristic of VDs of distinct antibodies; and
if appropriate, a lipophilic element, said lipophilic element being bonded to VD2a via a bond that is labile in a reducing medium, for example a disulfide bridge, or via said linker, said linker comprising a bond that is labile in a reducing medium or being bonded to VD2a via a bond that is labile in a reducing medium;
the second chimeric molecule, respectively B or A, comprises or consists of:
said polypeptide domain (i), this domain and the polypeptide domain (i) of the first chimeric molecule being characteristic of VDs of the same antibody, one of these domains being characteristic of a VD of the light chain of a given antibody and the other being characteristic of a VD of the heavy chain of the same antibody;
and the following domains, positioned in succession in the following order, starting from the C-terminal end of the polypeptide domain (i):
if appropriate, said linker; and
said nucleotide domain (ii), termed S2, capable of pairing into a double stranded structure with S1 in an aqueous medium, in particular a reducing medium, and
said half-body, termed HAB_{—}1, in which:
the polypeptide domain (i) of the chimeric molecules A and B is preferably characteristic of a VD of an antibody directed against a first antigen present on the surface of mammalian cells or directed against a viral antigen, for example against the outer envelope protein of a HIV virus; and
the polypeptide domain VD2a is preferably characteristic of a VD of an anti-retrotranscriptase antibody, in particular HIV-1 or HIV-2 anti-retrotranscriptase; or
a half-antibody, in which:
the first chimeric molecule, A or B, comprises or consists of:
said polypeptide domain (i), and the following domains, positioned in succession in the following order, starting from the C-terminal end of the polypeptide domain (i):
if appropriate, said linker;
the nucleotide domain (ii), termed S5;
if appropriate, said linker, said linker comprising, if appropriate, a bond that is labile in a reducing medium, for example a disulfide bridge, or being bonded via a bond that is labile in a reducing medium, for example a disulfide bridge, to:
a second nucleotide domain termed S4, which comprises or consists of a polynucleotide, in particular a DNA or a RNA, or an analog of a polynucleotide, in particular a PNA, a LNA, a methylphosphonate nucleic acid or a thioate nucleic acid, and which is capable of pairing into a double stranded structure with a complementary nucleotide domain termed S3, for example in a hydric medium, in particular in a reducing medium; and
if appropriate, said linker,
the domain S4 or said linker being bonded to the C-terminal end of a second polypeptide domain, termed VD2b, which is characteristic of a VD of a heavy chain or of a light chain of an antibody, VD2b and the polypeptide domain (i) of the first chimeric molecule being characteristic of VDs of distinct antibodies; and
if appropriate, a lipophilic element, said lipophilic element being bonded to VD2b via a bond that is labile in a reducing medium, for example a disulfide bridge, or via said linker, said linker comprising a bond that is labile in a reducing medium or being bonded to VD2a via a bond that is labile in a reducing medium; and
the second chimeric molecule, respectively B or A, comprises or consists of:
said polypeptide domain (i), this domain and the polypeptide domain (i) of the first chimeric molecule preferably being characteristic of VDs of the same antibody, one of these domains being characteristic of a VD of the light chain of a given antibody and the other being characteristic of a VD of the heavy chain of the same antibody,
and the following domains, positioned in succession in the following order, starting from the C-terminal end of the polypeptide domain (i):
if appropriate, said linker; and
said nucleotide domain (ii), termed S6, which is capable of pairing into a double stranded structure with S5 in an aqueous medium, in particular a reducing medium, said half-antibody, termed HAB_{—}2, in which
the polypeptide domain (i) of the chimeric molecules A and B is preferably characteristic of a VD of an antibody directed against a second antigen present on the surface of mammalian cells, for example against the CD4 protein, or directed against a viral antigen; and
the polypeptide domain VD2b is preferably characteristic of a VD of the same antibody as the VD2a domain, the VD2a or VD2b domain being characteristic of a VD of the light chain of a given antibody, for example an anti-retrotranscriptase antibody, and the other domain, respectively VD2b or VD2a, being characteristic of a VD of a heavy chain of the same antibody.

10. A particle, in particular a nanoparticle or Q-dot, onto which one or more half-antibodies as defined in claim 1, preferably via:
one or more bond(s) that are labile in a reducing medium, for example via a disulfide bridge; or
a PNA or a nucleic acid comprising a sequence that is complementary to a nucleic sequence of said half-antibody (for example complementary to the tag domain of the half-antibody).

11. A composition, in particular a pharmaceutical or therapeutic composition comprising, consisting or essentially consisting of one or more half-antibodies as defined in claim 1 and, if appropriate, a support, a diluent and/or a pharmaceutically acceptable vehicle.

12. A composition as claimed in claim 11, for use as a drug, in particular for the prevention and/or treatment of a cancer and/or a genetic disease, for example a myopathy, and/or an infectious disease, in particular a viral disease, more particularly an infection by a lentivirus, for example HIV-1 or HIV-2, and/or a bacterial infection, for example an infection by a bacterium that is resistant to antibiotics.

13. A kit comprising one or more half-antibodies as defined in claim 1, and if appropriate, instructions for use.

14. A kit as claimed in claim 13, for use as a drug, in particular for the prevention and/or treatment of a cancer and/or a genetic disease, for example a myopathy, and/or an infectious disease, in particular a viral disease, more particularly an infection by a lentivirus, for example HIV-1 or HIV-2, and/or a bacterial infection, for example an infection by a bacterium that is resistant to antibiotics.

15. A method for detecting and, if appropriate, quantifying one or more antigen(s) of interest that may be present in a sample, said method comprising or consisting of the following steps:
bringing antigens of the sample into contact with one or more half-antibodies as defined in the present application, in particular with the half-antibodies HAB_{—}1 and/or HAB_{—}2;
detecting, for example by PCR, any complexes that may be formed between said half-antibody or antibodies and one or more antigen(s) of the sample;
if appropriate, quantifying the antigen or antigens detected thereby, for example by PCR;
in which the antigens that may be present in the biological sample have been fixed onto a solid support.

16. A chip, characterized in that it comprises one or more half-antibodies as defined in claim 1, said half-antibody or half-antibodies being bonded to one or more hybridization units (or spots) of said chip, and preferably said half-antibody or said half-antibodies being such that:
the first chimeric molecule, A or B, of said half-antibody comprises or consists of:
said polypeptide domain (i) as, and the following domains, positioned in succession in the following order, starting from the C-terminal end of polypeptide domain (i):
if appropriate, a linker (for example a peptide linker);
said nucleotide domain (ii);
if appropriate, a linker (for example a nucleic linker), bonded, preferably via a bond that is labile in a reducing medium (for example a disulfide bridge) or via a bond that persists in a reducing medium (for example via a bond established with a maleimide acid), to a tag domain that is complementary to a nucleotide sequence of a hybridization unit on said chip; and
the second chimeric molecule, respectively B or A, of said half-antibody comprises or consists of:
said polypeptide domain (i), this domain and the polypeptide domain (i) of the first chimeric molecule preferably being characteristic of VDs of the same antibody, one of these domains being characteristic of a VD of the light chain of a given antibody and the other being characteristic of a VD of the heavy chain of the same antibody;
and the following domains, positioned in succession in the following order, starting from the C-terminal end of the polypeptide domain (i):
if appropriate, a linker (for example a peptide linker); and
said nucleotide domain (ii), capable of pairing in a hydric medium (in particular a reducing medium) into a double stranded structure with the nucleotide domain (ii) of the first chimeric molecule, respectively A or B.

17. A method for detecting and, if appropriate, quantifying one or more antigen(s) of interest that may be present in a sample, said method comprising or consisting of the following steps:
a) bringing one or more hybridization units (or spots) of a nucleic acid chip into contact with one (or more) half-antibodies as defined in claim 1, the half-antibody being capable of becoming attached to one or more hybridization unit(s) of said chip;
b) bringing hybridization units of the chip (or the hybridization units that have been brought into contact with the half-antibody in step a)) into contact with the proteins or antigens of the sample (or more generally with the sample);
c) detecting complexes that may be formed between the half-antibody attached to the chip and one or more antigen(s) of the sample;
d) if appropriate, quantifying the antigen(s) detected thereby.

18. A kit, in particular a kit appropriate for use in detecting and, if appropriate, quantifying one or more antigen(s) of interest that may be present in a sample, said kit comprising or consisting of:
a)—a nucleic acid chip; and
one or more half-antibodies as defined in; claim 1; and
if appropriate, instructions for use; or
b) a nucleic acid chip that comprises one or more hybridization units (or spots) on which a half-antibody as defined in claim 1 has been hybridized; and
if appropriate, instructions for use.

19. An enzyme or PNAzyme formed by a structure comprising or consisting of:
a PNA sequence, bonded to:
a peptide sequence, bonded to:
another PNA sequence;
the two PNA sequences possibly being identical or different, in which the peptide sequence preferably constitutes a catalytic domain of an enzyme, in particular of a nuclease, preferably a DNAse.
